(19) United States
(12) Patent Application Publication
(10) Pub. No.: US 2022/0251603 A1
(43) Pub. Date: Aug. 11, 2022

(54) SYSTEMS AND METHODS FOR GENE THERAPY VIA ADMINISTRATION OF GENETICALLY MODIFIED VIRAL VECTORS

(71) Applicant: BioViva USA, Inc., Bainbridge Island, WA (US)

(72) Inventors: Elizabeth Louise Parrish, Bainbridge Island, WA (US); Anca Selariu, San Diego, CA (US); Dabbu Kumar Jaijyan, Newark, NJ (US); Hua Zhu, Newark, NJ (US)

(21) Appl. No.: 17/592,803

(22) Filed: Feb. 4, 2022

Related U.S. Application Data
(60) Provisional application No. 63/305,836, filed on Feb. 2, 2022.
(60) Provisional application No. 63/146,538, filed on Feb. 5, 2021.
(60) Provisional application No. 63/188,652, filed on May 14, 2021.

(51) Int. Cl. C12N 15/86; C12N 9/12; C07K 14/71; A61P 17/14; A61P 3/08; A61P 39/00

(52) U.S. Cl. CPC C12N 15/86; C12N 9/1276; C12Y 207/07049; C07K 14/71; A61K 38/00; A61P 3/08; A61P 39/00; C12N 2710/16143; C07K 2319/43; A61P 17/14

(57) ABSTRACT

Gene therapy vectors can include a cytomegalovirus vector encoding one or more therapeutic donor genes. These vectors can be used in exemplary gene therapy methods for maintaining or improving one or more aspects of a recipient's physiological wellness and/or longevity. The recombinant viral vector can be administered or received intranasally or as an injectable therapeutic (singly or as a serial set of administrations) to beneficially cause one or more of the following salubrious effects in the patient: increased longevity, inhibited muscle degeneration, increases mitochondrial health, prevention of age-related hair loss, and/or increased blood glucose tolerance.

### CROSS-REFERENCE TO RELATED APPLICATIONS

This application claims the benefit of and priority to: U.S. Provisional Application No. 63/146,538 filed Feb. 5, 2021; U.S. Provisional Application No. 63/188,652 filed May 14, 2021; and U.S. Provisional Application No. 63/305,836 filed Feb. 2, 2022, each of which is incorporated herein by reference in its entirety.

### BACKGROUND

### Technical Field

This disclosure generally relates to gene therapy methods and compositions used in the same. More specifically, the present disclosure relates to genetically-modified viral vectors and methods for administering the same for the purpose of maintaining or improving one or more aspects of a recipient's physiological wellness and/or longevity.

### Related Technology

Progress in the study of genetics and cellular biology over the past three decades has greatly enhanced our ability to describe the molecular basis of many human diseases. Molecular genetic techniques have been particularly effective. These techniques have allowed the isolation of genes associated with common inherited diseases that result from a lesion in a single gene such as ornithine transcarbamylase (OTC) deficiency, cystic fibrosis, hemophilias, immunodeficiency syndromes, and others—as well as those that contribute to more complex diseases such as cancer. As a result, gene therapy, defined as the introduction of genetic material into a cell in order to either change its phenotype or genotype, has been intensely investigated over the last few decades.

For effective gene therapy, it is necessary to deliver therapeutic genes to relevant cells in vivo at high efficiency, to express the therapeutic genes for prolonged (therapeutic) periods of time, and to ensure that the transduction events do not have deleterious effects. To meet these criteria, a variety of vector systems have been evaluated. These systems include viral vectors, such as retroviruses (including lentiviruses), adenoviruses, adeno-associated viruses, and herpes simplex viruses, and non-viral systems such as liposomes, molecular conjugates, and other particulate vectors. Although viral systems have been efficient in laboratory studies, none have yet been definitively curative in human clinical applications.

In general, gene therapy vectors can be classified as two main types—viral and non-viral. The most commonly used viral gene therapy systems are retroviral vectors and adenoviral vectors, in part for historical reasons and in part because they have been relatively straightforward to make in clinically useful quantities. Retroviral and adenoviral vectors have both been used in some instances within the clinic, and some clinical trials have also been conducted using Adeno-associated viral vectors, rhabdoviruses, herpes viral vectors, and vectors based on vaccinia virus or poxviruses. These viruses have various strengths and weaknesses but are all relatively efficient at delivering genes to target tissues. Limitations include difficulties in making sufficient quantities, inability to accurately target the gene delivery in vivo, or toxic or immunological side effects of viral gene products. However, it should be noted that even with relatively efficient viral vectors, it is not reasonable with conventional technology to expect that a gene can be delivered to every affected cell (or desired target cell).

Non-viral gene therapy systems include naked DNA, DNA formulated in liposomes, DNA formulated with polycation condensing agents or hybrid systems, and DNA conjugated with peptides or proteins, such as single chain antibodies, to target them to specific tissues. These systems are more amenable to building in rational regulated steps to accomplish a long in vivo half-life, delivery to the target cell/tissue of interest, and entry into the cytoplasm and nucleus of targeted cells with attendant expression. Although there are possible solutions to each of these issues, they have not yet been efficiently combined, and efficiency of gene transfer in vivo remains elusive. Thus, for these systems also, it is not reasonable at present to expect that a gene can be delivered to every desired target cell or to otherwise provide an organism-wide salubrious effect.

In an effort to allow some kind of amplification of the gene delivery events, prior efforts have attempted stimulation of the immune system, various forms of bystander effects, spread of apoptosis, antiangiogenic effects, pro-coagulant effects, replication of competent viral vectors, and other mechanisms.

Adenoviridae is a family of DNA viruses first isolated in 1953 from tonsils and adenoidal tissue of children. Six sub-genera (A, B, C, D, E, and F) and more than 49 serotypes of adenoviruses have been identified as infectious agents in humans. Although a few isolates have been associated with tumors in animals, none have been associated with tumors in humans. The adenoviral vectors most often used for gene therapy belong to the subgenus C, serotypes 2 or 5 (Ad2 or Ad5). These serotypes have not been associated with tumor formation. Infection by Ad2 or Ad5 results in acute mucous-membrane infection of the upper respiratory tract, eyes, lymphoid tissue, and mild symptoms similar to those of the common cold. Exposure to C-type adenoviruses is widespread in the population with the majority of adults being seropositive for this type of adenovirus.

Adenovirus virions are icosahedrons of 65-80 nm in diameter containing 13% DNA and 87% protein. The viral DNA is approximately 36 kb in length and is naturally found in the nucleus of infected cells as a circular episome held together by the interaction of proteins covalently linked to each of the 5′ ends of the linear genome. The ability to work with functional circular clones of the adenoviral genome greatly facilitated molecular manipulations and allowed the production of replication defective vectors.

Two aspects of adenoviral biology have been critical in the production of commonly used replication incompetent adenoviral vectors. First is the ability to have essential regulatory proteins produced in trans, and second is the inability of adenovirus cores to package more than 105% of the total genome size. The first was originally exploited by the generation of 293 cells, a transformed human embryonic kidney cell line with stably integrated adenoviral sequences from the left-hand end (0-11 map units) comprising the E1 region of the viral genome. These cells provide the E1A gene product in trans and thus permit production of virions with genomes lacking E1A. Such virions are considered replication deficient since they cannot maintain active replication in cells lacking the E1A gene (although replication may occur at high vector concentrations). 293 cells are permissive for the production of these replication deficient vectors and have been utilized in many, if not all, approved gene therapy protocols that use adenoviral vectors.

The second was exploited by creating backbones that exceed the 105% limit to force recombination with shuttle plasmids carrying the desired transgene. Most currently used adenoviral vector systems are based on backbones of subgroup C adenovirus, serotypes 2 or 5. Deleting regions E1/E3 alone or in combination with E2/E4 produced first- or second-generation replication-defective adenoviral vectors, respectively. As mentioned above, the adenovirus virion can package a genome up to 105% of the size of the wild-type genome, allowing for the insertion of approximately 1.8 kb of additional heterologous DNA. The deletion of E1 sequences adds another 3.2 kb, while deletion of the E3 region provides an additional 3.1 kb of foreign DNA space. Therefore, E1 and E3 deleted adenoviral vectors provide a total capacity of approximately 8.1 kb of heterologous DNA sequence packaging space.

Adenoviruses have been extensively characterized and make attractive vectors for gene therapy because of their relatively benign symptoms (even as wild-type infections), their ease of manipulation in vitro, the ability to consistently produce high titer purified virus, their ability to transduce quiescent cells, and their broad range of target tissues. In addition, adenoviral DNA is not incorporated into host cell chromosomes, minimizing concerns about insertional mutagenesis or potential germ line effects. This has made them attractive vectors for tumor gene therapy protocols and other protocols in which transient expression may be desirable. However, these vectors are not very useful for metabolic diseases and other application for which long-term expression may be desired. Human subgroup C adenoviral vectors lacking all or part of E1A and E1B regions have been evaluated in Phase I clinical trials that target cancer, cystic fibrosis, and other diseases without major toxicities being described. A major exemption to the safety of these vectors was the death of a young man that received a very large dose of E1, E4 deleted vector directly into the hepatic artery. The large bolus dose of adenoviral virions led to liver toxicity, a DIC-like response, and ultimately respiratory distress and death.

The use of “replication conditional” adenoviruses for cancer therapy has shown some effects in clinical studies. “Replication conditional” vectors or viruses either lack a portion of the genome that is important for replication in “normal” cells, but less critical in the target cells, or contain regulatory elements that target specific tissues (e.g., a tissue specific promoter for the expression of the E1A, E1B, E2, or E4 regions of the virus). A major concern for the efficacy of these vectors, as for replication deficient adenoviral vectors, is the original host response to the delivered virions and the limited ability for repeated administration due to the host's antiviral immune response, which diminishes the effectiveness of using these viral vectors in primary and subsequent applications. To address the issue of immune neutralization of viral vectors, it is advantageous to deliver the necessary nucleic acid sequences for the production of the virions by a non-viral method, especially if these can be delivered in a targeted method.

Retroviruses are one of the most intensely scrutinized group of viruses in recent years. The Retroviridae family has traditionally been subdivided into three subfamilies largely based on the pathogenic effects of infection, rather than phylogenetic relationships. The common names for the subfamilies are tumor- or onco-viruses, slow- or lenti-viruses and foamy- or spuma-viruses. The latter have not been associated with any disease and are the least well known. Retroviruses are also described based on their tropism: ecotropic, for those which infect only the species of origin (or closely related species); amphotropic, for those which have a wide species range normally including humans and the species of origin; and xenotrophic, for those which infect a variety of species but not the species of origin.

Tumor viruses comprise the largest of the retroviral subfamilies and have been associated with rapid or slow tumor production. Onco-viruses are sub-classified as types A, B, C, or D based on the virion structure and process of maturation. Most retroviral vectors developed to date belong to the C type of this group. These include the Murine leukemia viruses and the Gibbon ape virus and are relatively simple viruses with few regulatory genes. Like most other retroviruses, C type based retroviral vectors disadvantageously require target cell division for integration and productive transduction.

An important exception to the requirement for cell division is found in the lentivirus subfamily. The human immunodeficiency virus (HIV), the most well-known of the lentiviruses and etiologic agent of acquired immunodeficiency syndrome (AIDS), was shown to integrate in non-dividing cells. Although the limitation of retroviral integration to dividing cells may be a safety factor for some protocols, such as cancer treatment protocols, it is probably the single most limiting factor in their utility for the treatment of inborn errors of metabolism and degenerative traits.

Examples of retroviruses are found in almost all vertebrates, and despite the great variety of retroviral strains isolated and the diversity of diseases with which they have been associated, all retroviruses share similar structures, genome organizations, and modes of replication. Retroviruses are enveloped RNA viruses approximately 100 nm in diameter. The genome consists of two positive RNA strands with a maximum size of around 10 kb. The genome is organized with two long terminal repeats (LTR) flanking the structural genes gag, pol, and env. The presence of additional genes (regulatory genes or oncogenes) varies widely from one viral strain to another. The env gene codes for proteins found in the outer envelope of the virus. These proteins convey the tropism (species and cell specificity) of the virion. The pol gene codes for several enzymatic proteins important for the viral replication cycle. These include the reverse transcriptase, which is responsible for converting the single stranded RNA genome into double stranded DNA, the integrase which is necessary for integration of the double stranded viral DNA into the host genome, and the proteinase which is necessary for the processing of viral structural proteins. The gag, or group specific antigen gene, encodes the proteins necessary for the formation of the virion nucleocapsid. The necessity of these genes for the productive infection and life cycle of retroviral vectors limits the nucleotide length and/or number of genes that can be successfully employed.

While recombinant retroviruses are considered efficient vectors for the stable transfer of genetic material into the genome of actively replicating mammalian cells, the retroviral vector is a non-replicating delivery system with the capacity to encode a mere 8 kb of genetic information. To assemble and propagate a recombinant retroviral vector, the missing viral gag-pol-env functions must be supplied in trans.

Since their development in the early 1980's, vectors derived from type C retroviruses represent some of the most useful gene transfer tools for gene expression in human and mammalian cells. Their mechanisms of infection and gene expression are well understood. The advantages of retroviral vectors include their relative lack of intrinsic cytotoxicity and their ability to integrate into the genome of actively replicating cells. However, there are a number of limitations for retroviruses as a gene delivery system including a limited host range, instability of the virion, a requirement for cell replication, and the limited genomic/capsid space, which restricts the size and number of cargo genes.

Although amphotropic retroviruses have a broad host range, some cell types are relatively refractory to infection. One strategy for expanding the host range of retroviral vectors has been to use the envelope proteins of other viruses to encapsidate the genome and core components of the vector. Such pseudotyped virions exhibit the host range and other properties of the virus from which the envelope protein was derived. For example, the envelope gene product of a retrovirus can be replaced by VSV-G to produce a pseudotyped vector able to infect cells refractory to the parental vector. While retroviral infection usually requires specific interaction between the viral envelope protein and specific cell surface receptors, VSV-G interacts with a phosphatidyl serine and possibly other phospholipid components of the cell membrane to mediate viral entry by membrane fusion. Since viral entry is not dependent on the presence of specific protein receptors, VSV has an extremely broad host-cell range. In addition, VSV can be concentrated by ultracentrifugation to titers greater than 10⁹ plaque forming units per mL with minimal loss of infectivity, while attempts to concentrate amphotropic retroviral vectors by ultracentrifugation or other physical means has resulted in significant loss of infectivity with only minimal increases in final titer.

However, since VSV-G protein mediates cell fusion it is toxic to cells in which it is expressed. This has led to technical difficulties for the production of stable pseudotyped retroviral packaging cell lines. One approach for production of VSV-G pseudotyped vector particles has been by transient expression of the VSV-G gene after DNA transfection of cells that express a retroviral genome and the gag/pol components of a retrovirus. Generation of vector particles by this method is cumbersome, labor intensive, and not easily scaled up for extensive experimentation.

Others have produced VSV-G pseudotyped retroviral packaging through adenovirus-mediated inducible gene expression. For example, tetracycline-controllable expression has been used to generate recombinant adenoviruses encoding the cytotoxic VSV-G protein. A stably transfected retroviral genome was rescued by simultaneous transduction with three recombinant adenoviruses: one encoding the VSV-G gene under control of the tet promoter, another the retroviral gag/pol genes, and a third encoding the tetracycline transactivator gene. This resulted in the production of VSV-G pseudotyped retroviral vectors. Although both of these systems produce pseudotyped retroviruses, both are unlikely to be amenable to clinical applications that demand reproducible, certified vector preparation.

Another limitation for the use of retroviral vectors for human gene therapy applications has been their short in vivo half-life. This is partly due to the fact that human and non-human primate sera rapidly inactivate type C retroviruses. Viral inactivation occurs through an antibody-independent mechanism involving the activation of the classical complement pathway. The human complement protein Clq was shown to bind directly to MLV virions by interacting with the transmembrane envelope protein p15E. An alternative mechanism of complement inactivation has been suggested based upon the observation that surface glycoproteins generated in murine cells contain galactose-α-(1,3)-galactose sugar moieties. Humans and other primates have circulating antibodies to this carbohydrate moiety. These anticarbohydrate antibodies are able to fix complement, which leads to subsequent inactivation of murine retroviruses and murine retrovirus producer cells by human serum. Therefore, inactivation of retroviral vectors by complement in human serum is determined by the cell line used to produce the vectors and by the viral envelope components.

Prior efforts of achieving a better gene therapy system have included the use of a multiple adenoviral vector system to transiently transduce cells to produce retroviral progeny. An adenoviral vector encoding a retroviral backbone (the LTRs, packaging sequence, and a reporter gene) and another adenoviral vector encoding all of the trans acting retroviral functions (the CMV promoter regulating gag, pol, and env) has been shown to accomplish in vivo gene transfer to target parenchymal cells at high efficiency, rendering them transient retroviral producer cells. Athymic mice xenografted orthotopically with the human ovary carcinoma cell line SKOV3 and then challenged intraperitoneally with the two adenoviral vector systems demonstrated the concept that adenoviral transduction had occurred with the in situ generation of retroviral particles that stably transduced neighboring cells in the target parenchyma. While these experimental systems have established the foundation that adenoviral vectors may be utilized to render target cells transient retroviral vector producer cells, they are unlikely to be easily amenable to clinical applications that demand reproducible, certified vector preparation because of the stochastic nature for multiple vector transduction of single cells in vivo.

Adenovirus-associated viruses are simple DNA containing viruses often requiring the function of other viruses (e.g. adenoviruses or herpes viruses) for complete replication efficiency. The virion is composed of a rep and cap gene flanked by two inverted terminal repeats (ITRs). These vectors have the ability to integrate into the cellular genome for stable gene transfer. However, a major hinderance to further use of these vectors has been the ability to produce them in large-scale in vitro. The major obstacles to this endeavor is the toxic cellular effects of the rep and needed helper-virus genes. Examples of production methods for AAV vectors include co-transfection of plasmids delivering the ITR flanked gene of interest with a rep-cap expression cassette and the helper-virus genes and co-delivery of the ITR-flanked gene of interest along with helper-virus genes to cells stably expressing rep-cap, delivery of a chimeric virus vector, such as a herpes virus vector, with all the necessary components.

Deficiencies in the art regarding methods of utilizing adenoviral, retroviral, and adeno-associated elements for stable delivery of a therapeutic gene include lack of a single vector. The requirement for multiple vectors dictates that more antibiotics are used, which is more costly and furthermore undesirable, given the increasing number of strains which are becoming resistant to commonly used antibiotics. In addition, the use of multiple vectors gives reduced efficiency, since more than one transduction event into an individual cell is required, which statistically occurs at a reduced amount compared to requirement for one transduction event. Other notable deficiencies in the art include rapid clearance and host immunity to the viral vectors, limiting their prolonged use, possible oncogenic effects from viral integration, limited cellular tropisms, and the limited size and/or number of genes that can be delivered in a single vector.

Accordingly, there are a number of disadvantages with gene therapy approaches that can be addressed to solve a long-felt and unmet need in the art.

### BRIEF DESCRIPTION OF THE DRAWINGS

In order to describe the manner in which the above recited and other advantages and features of the disclosure can be obtained, a more particular description of the disclosure briefly described above will be rendered by reference to specific embodiments thereof, which are illustrated in the appended drawings. It is appreciated that these drawings depict only typical embodiments of the disclosure and are not therefore to be considered to be limiting of its scope. The disclosure will be described and explained with additional specificity and detail through the use of the accompanying drawings in which:

FIGS. 1A-1D illustrate the construction and characterization of recombinant MCMV_{TERT} and MCMV_{FS344} (also referred to herein as simply MCMV_{FST}) expressing mTERT and mFS344 (also referred to herein as simply mFST). FIG. 1A is a graphical representation of the location of mTERT and mFS344 genes insertion in the MCMV genome. FIG. 1B illustrates a Western blot analysis of mTERT and mFS344 in MCMV_{TERT} and MCMV_{FS344} infected cells as compared to cells infected with respective non-recombinant MCMV (WT). FIG. 1C illustrates a growth curve of MCMV_{TERT} and MCMV_{FS344} viruses in cell culture as compared to wild-type, non-recombinant MCMV (WT). FIG. 1D illustrates images obtained using IVIS during an in vivo luciferase assay of 20 months old C57BL/6J mice that were uninfected, infected with wild-type MCMV virus, or infected with MCMV_{TERT} or MCMV_{FS344} recombinant virus.

FIGS. 2A-2D are various graphs of data illustrating improved health of mice infected with recombinant MCMV_{TERT} or MCMV_{FS344} virus. In particular, FIG. 2A illustrates the results of a glucose tolerance test (GTT) in mice treated intraperitoneally (IP) or intranasally (IN) with WT-MCMV, with recombinant MCMV_{TERT} virus, or with recombinant MCMV_{FS344} virus, as compared to untreated (UN) controls. FIG. 2B is a bar graph illustrating the calculated area under the curve (AUC) in the GTT assay of FIG. 2A. FIG. 2C is a bar graph illustrating the results of a 3-minute beaker test in the respective groups, and FIG. 2D is a bar graph illustrating the results of a beam test for mice tested in each infected and uninfected group.

FIG. 3A is a graph illustrating mTERT mRNA levels within different organ tissues of mice 5 days post infection with MCMV_{Luc-TERT}.

FIG. 3B is a graph comparing TERT and FST mRNA levels in different organs of WT, MCMV_{TERT}, and MCMV_{FST} treated mice by qPCR, with fold increase in MCMV_{TERT}, and MCMV_{FST} treated mice over WT treated mice indicated numerically on the top of each bar.

FIG. 3C illustrates the determination of relative telomere length in different organs of treated and untreated groups at 24-months-old mice. An 8-months-old mouse was also measured. The qPCR was performed on the genomic DNA using specific telomeric primers. A pair of 36B4 gene primers was used as a single copy gene for normalization.

FIGS. 4A-C illustrates detection of mTERT and mFST recombinant proteins by ELISA in the sera of WT, MCMV_{TERT}, and MCMV_{FST} treated (IP or IN) 24-month-old mice. FIGS. 4A and 4B illustrate detected levels over four days. FIG. 4C illustrates detection of recombinant mTERT by ELISA over a one-month period of time, as detected in the serum of 8-month-old mice infected or uninfected with recombinant MCMV_{TERT} virus. In each of FIGS. 4A-4C, mock treated mice were used as negative control. Each data point represents the average value of three mice. Error bars represent standard deviations.

FIGS. 5A-5D provide evidence that treatment with recombinant MCMV_{TERT} and MCMV_{FS344} virus improves quality of life in treated mice. FIGS. 5A and 5B are exemplary images of 26-month-old C57BL/6J mice exhibiting hair loss as an uninfected control (FIG. 5A) and following infection by wild-type MCMV virus (FIG. 5B). FIGS. 5C and 5D are exemplary images of 26-month-old C57BL/6J mice exhibiting no loss of hair (or hair retention) following infection with recombinant MCMV_{TERT} (FIG. 5C) and MCMV_{FS344} (FIG. 5D) virus.

FIG. 6 illustrates a graph of the average body weight of mice in the untreated and treated cohorts; each cohort being tested within two different treatment sets.

FIGS. 7A and 7B illustrates survivorship for mice in the untreated and treated cohorts; each cohort being tested within two different treatment sets.

FIGS. 8A-8C illustrate an analysis of mouse heart muscle by electron microscopy. FIG. 6A illustrates electron micrographs of heart muscle taken from 6-month old mice (young) and from aging mice within the uninfected control (UN), wild-type-MCMV-infected control (MCVM), MCMV_{TERT}-infected cohort, and MCMV_{FS344}-infected cohort (scale bar=500 nm). From these micrographs (N=20 in each group), the number of mitochondria within connected cristae and the density of mitochondria within mouse heart muscle is calculated (FIGS. 6B and 6C, respectively). Values are mean±S.E., p<0.05 using the unpaired Student's t-test.

FIGS. 9A-9C illustrate an analysis of mouse skeletal muscle by electron microscopy. FIG. 7A illustrates electron micrographs of skeletal muscle taken from 6-month old mice (young) and from aging mice within the uninfected control (UN), wild-type-MCMV-infected control (MCVM), MCMV_{TERT}-infected cohort, and MCMV_{FS344}-infected cohort (scale bar=500 nm). From these micrographs (N=20 in each group), the number of mitochondria within connected cristae and the density of mitochondria within mouse skeletal muscle is calculated (FIGS. 7B and 7C, respectively). Values are mean±S.E., p<0.05 using the unpaired Student's t-test.

FIG. 10 illustrates the results of mouse skeletal muscle tissue homogenates obtained from uninfected mice and mice infected with recombinant MCMV expressing mouse TERT and mouse FS344 genes and probed by Western blot for various mitochondrial and autophagy markers. PGC1α and TFAM represent transcriptional factors for mitochondrial biogenesis; Complex I, Complex II, Complex III, and Complex V represent markers for mitochondria function; and LCI, LC3II, and p62 represent autophagy markers. GAPDH was used as a loading control.

FIG. 11 illustrates the results of mouse heart muscle tissue homogenates obtained from uninfected mice and mice infected with recombinant MCMV expressing mouse TERT and mouse FS344 genes and probed by Western blot for various mitochondrial and autophagy markers. PGC1α represents transcriptional factors for mitochondrial biogenesis; Complex I, Complex II, Complex III, and Complex V represent markers for mitochondria function; and LC3I, LC3II, and p62 represent autophagy markers. GAPDH was used as a loading control.

FIGS. 12A-12E illustrate example bacterial artificial chromosome (BAC) constructs that may be utilized to generate the CMV vectors disclosed herein, the constructs including one or more therapeutic genes suitable for administration to human subjects. FIG. 12A shows an example construct that includes human telomerase reverse transcriptase (hTERT) as an exogenous insert. FIG. 12B shows an example construct that includes human follistatin-344 (hFST) as an exogenous insert. FIG. 12C shows an example construct that includes klotho (KL or KLOTHO) as an exogenous insert. FIG. 12D shows an example construct that includes Dsup as an exogenous insert, and FIG. 12E shows an example construct that includes PGC1α as an exogenous insert.

FIGS. 13A-13F illustrate example BAC constructs that may be utilized to generate the CMV vectors disclosed herein, each construct including multiple therapeutic genes suitable for administration to human subjects. FIG. 13A shows an example construct that includes both hTERT and hFST as exogenous inserts. FIG. 13B shows an example construct that includes both hFST and KLOTHO as exogenous inserts. FIG. 13C shows an example construct that includes both hTERT and KLOTHO as exogenous inserts. FIG. 13D shows an example construct that includes hTERT, hFST, and PGC1α as exogenous inserts. FIG. 13D shows an example construct that includes hTERT, hFST, and KLOTHO as exogenous inserts. FIG. 13E shows an example construct that includes hTERT, hFST, and Dsup as exogenous inserts, and FIG. 13F shows an example construct that includes hTERT, hFST, and PGC1α as exogenous inserts.

FIG. 14 illustrates a method for manufacturing a CMV-hFST recombinant BAC via homologous recombination.

FIG. 15 illustrates a method for manufacturing a CMV-hTERT recombinant BAC via homologous recombination.

FIG. 16 illustrates a method for manufacturing a CMV-hFST+hTERT recombinant BAC via homologous recombination.

FIG. 17 illustrates a method for manufacturing a CMV-hFST+hTERT+KL recombinant BAC via homologous recombination.

### DETAILED DESCRIPTION

### Selected Terms & Definitions

Before describing various embodiments of the present disclosure in detail, it is to be understood that any headings used herein are for organizational purposes only and are not meant to be used to limit the scope of the description or the claims. It should be further appreciated that various aspects of the present disclosure, including devices, systems, and methods, may be illustrated with reference to one or more embodiments or implementations, which are exemplary in nature. As used herein, the term “exemplary” means “serving as an example, instance, or illustration,” and should not necessarily be construed as preferred or advantageous over other embodiments disclosed herein. In addition, reference to an “implementation” of the present disclosure or invention includes a specific reference to one or more embodiments thereof, and vice versa, and is intended to provide illustrative examples without limiting the scope of the invention, which is indicated by the appended claims rather than by the following description. This disclosure is not limited to the parameters of the particularly exemplified systems, methods, apparatus, products, processes, and/or kits, which may, of course, vary. Similarly, while specific language will be used herein to describe the exemplary embodiments, it will be understood that no limitation of the scope of the disclosure is thereby intended. Rather, it is to be understood that the language used to describe the exemplary embodiments is illustrative only and is not to be construed as limiting the scope of the disclosure (unless such language is expressly described herein as essential).

Unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

It is noted that in this disclosure and particularly in the claims and/or paragraphs, terms such as “comprises,” “comprised,” “comprising,” and the like can have the meaning attributed to it in U.S. Patent law (e.g., they can mean “includes,” “included,” “including,” and the like); and that terms such as “consisting essentially of” and “consists essentially of” have the meaning ascribed to them in U.S. Patent law (e.g., they allow for elements not explicitly recited, but exclude elements that are found in the prior art or that affect a basic or novel characteristic of the claimed embodiments). It should, therefore, be appreciated that the terms “including,” “having,” “involving,” “containing,” “characterized by,” as well as variants thereof (e.g., “includes,” “has,” “involves,” “contains,” etc.), and similar terms as used herein, including within the claims, shall be inclusive and/or open-ended, shall have the same meaning as the word “comprising” and variants thereof (e.g., “comprise” and “comprises”), and do not exclude additional un-recited elements or method steps, illustratively.

As used throughout this application the words “can” and “may” are used in a permissive sense (i.e., meaning having the potential to), rather than the mandatory sense (i.e., meaning must). Further, it will be noted that, the singular forms “a,” “an,” and “the”—as used in this specification and the appended claims—include plural referents unless the context clearly dictates otherwise. Thus, for example, reference to a singular referent (e.g., “widget”) includes one, two, or more referents. Similarly, reference to a plurality of referents should be interpreted as comprising a single referent and/or a plurality of referents unless the content and/or context clearly dictate otherwise. For example, reference to referents in the plural form (e.g., “widgets”) does not necessarily require a plurality of such referents. Instead, it will be appreciated that independent of the inferred number of referents, one or more referents are contemplated herein unless stated otherwise. The use of “or” means “and/or” unless stated otherwise. For illustration purposes, but not as a limitation, “X and/or Y” can mean “X” or “Y” or “X and Y”.

All literature cited in this specification, including but not limited to, patents, patent applications, articles, books, and treatises are expressly incorporated by reference in their entirety for any purpose. In the event that any of the incorporated literature contradicts any term defined herein, this specification controls.

It should be appreciated that the disclosed treatment methods can be from the perspective of the healthcare practitioner or from the patient's perspective. For example, the exemplary embodiment above that provides for the administration of a therapeutically effective amount of one or more recombinant viral vectors to a patient in need thereof, is provided from the perspective of a healthcare provider. This same method act can alternatively be expressed from the patient's perspective—e.g., receiving a therapeutically effective amount of one or more recombinant viral vectors. The same or analogous modification can be applied to any of the recited method acts disclosed herein, unless explicitly stated otherwise, to cast a disclosed method from the perspective of a healthcare provider or from the patient/subject themselves.

Donor genes described herein may synonymously be referred to as exogenous genes, exogenous donor genes, insert genes, and the like. Donor genes may be referred to by the name of the gene itself, or by the name of the protein for which the gene encodes. For example, the Oct-4 protein is technically encoded by the POU5f1 gene, but it will be understood that the same gene may be referred to herein as the Oct-4 gene.

### Viral Vectors

As provided above, there are a number of disadvantages with current gene therapy approaches that can be addressed to solve a long felt and unmet need in the art. In particular, none of the traditional retroviral, adenoviral, or adeno-associated gene therapy systems are capable of long-term, full-body expression of their payload in a manner that can promote the maintenance or improvement of aspect(s) of the recipient's physiological wellness and/or longevity. Instead, most, if not all, of the aforementioned gene therapy systems are directed to permanent correction of physiological or genetic defects within the targeted cells (e.g., via integration of the genetic payload at the target site) or are otherwise engineered to target tumors or provide cancer therapies. There is an apparent lack of solutions that address the many symptoms, effects, and/or disorders associated with aging.

Embodiments of the present disclosure solve one or more of the noted problems in the art. For example, a recombinant cytomegalovirus (CMV) vector is disclosed that encodes one or more therapeutic genes, and these therapeutic tools can be used to promote the maintenance or improvement of a recipient's physiological wellness and/or longevity. For example, the disclosed recombinant CMV vectors can be safely administered to a patient (e.g., intranasally and/or as an injectable preparation) and thereby deliver gene therapy to multiple organs with long-lasting benefits and no carcinogenicity or any other observable detrimental side effects. As a result, the disclosed vectors and associated treatment methods can increase longevity of patients by at least 10%, preferably at least 20%, and in some embodiments, by at least 30%. Additionally, the disclosed treatment methods can surprisingly and unexpectedly increase blood glucose tolerance, retain, or increase muscle mass, increase physical coordination, increase mitochondrial biogenesis in heart and skeletal muscle, increase autophagy, promote hair retention (or prevent age-related hair loss), or combinations thereof.

These salubrious effects are unexpected. For example, with respect to vector selection, CMV is a ubiquitous virus that is present in over 60% of the population. Following primary infection, CMV persists for the life span of the host, and although CMV is generally benign in healthy individuals, the virus can cause devastating disease in immunocompromised populations resulting in high morbidity and mortality. CMV is also one of the most immunogenic viruses known. High antibody titers are directed against numerous viral proteins during primary infection of healthy individuals. In addition, a large proportion of the host T-cell repertoire is also directed against CMV antigens, with 5 to 10-fold higher median CD4+ T-cell response frequencies to CMV than to acute viruses (e.g., measles, mumps, influenza, adenovirus) or even other persistent viruses such as herpes simplex and varicella-zoster viruses. A high frequency of CD8+ T-cell responses to defined CMV epitopes or proteins is also commonly observed. In a large-scale human study quantifying CD4+ and CD8+ T-cell responses to the entire CMV genome, the mean frequencies of CMV-specific CD4+ and CD8+ T-cells exceeded 10% of the memory population for both subsets, and in some individuals, CMV-specific T-cells account for >25% of the memory T-cell repertoire.

Paradoxically, the robust immune response to CMV is unable to either eradicate the virus from healthy infected individuals or confer protection against re-infection. This ability of CMV to escape eradication by the immune system and to re-infect the seropositive host has long been believed to be linked to the multiple viral immunomodulators encoded by the virus. The HCMV US6 family of proteins are the most extensively studied of these immunomodulators. At least four different genes, US2, US3, US6, and US11 are known to interfere with assembly and transport of MHC-I molecules.

As a result, CMV-based vectors expressing heterologous antigens do not induce cytotoxic T-cells directed against immunodominant epitopes of those heterologous antigens. This is particularly disadvantageous if the CMV-based vector is engineered as a vaccine or tumor-specific therapy as it limits the efficacy of the T-cells raised by such a CMV-based vaccine to protect against infection by a pathogen or to otherwise mount a cellular immune response against a tumor. On the other hand, while CMV-based vectors are poor candidates for vaccine development, the host's continued susceptibility to infection provides a unique opportunity for the serial administration of gene therapy—insofar as the vector is not toxic and the payload is not carcinogenic.

CMV can infect different cell types in the body and is thus able to deliver the target proteins to numerous cell types. More specifically, CMV can infect fibroblast, hepatocytes, endothelial cells, macrophages, epithelial cells, lymphocytes, retinal pigment epithelial cells, and cells of the gastrointestinal tract. Therefore, CMV can infect and deliver its target antigens to different cell types in various organs of the body. Moreover, CMV can overcome the pre-existing immunity to efficiently express the protein of interest. Furthermore, CMV has a large genome size owing to its tremendous capacity to incorporate multiple genes and express them simultaneously.

As discussed in more detail below, a BAC engineering method may be used to generate a recombinant CMV containing one or more desired therapeutic donor genes. Certain BAC methods, in relation to MCMV vectors with mouse telomerase reverse transcriptase (mTERT) gene, are described in Qiyi Tang, B. S., Hua Zhu. Protocol of a Seamless Recombination with Specific Selection Cassette in PCR-Based Site-Directed Mutagenesis. Applied Biological Engineering—Principles and Practice, 461-478 (2012).

### Vector Payloads

The recombinant CMV vectors disclosed herein may be configured to carry payloads (also referred to herein as donor genes, exogenous genes, inserts, and the like), that include one or more of: the human telomerase reverse transcriptase (hTERT) gene, the human follistatin-344 (hFST) gene, the klotho (KL) gene, the damage suppressor (Dsup) gene, the peroxisome proliferator-activated receptor gamma coactivator 1-alpha (PGC-1-α) gene (i.e., the PPARGC1A gene), and combinations thereof.

With respect to the disclosed payloads, the TERT gene is used to transcribe a catalytic component of telomerase enzyme, telomerase reverse transcriptase (TERT), which plays a major role in telomerase activation. Telomeres are short-repeated DNA segments (5′-TTAGGG-3′ in vertebrates) at chromosome ends, incompletely replicated during cell divisions. They protect our genetic material by acting as a chromosome cap, keeping them from binding to each other or breaking down. Once telomeres become too short, cells cease dividing and undergo apoptosis. Telomerase is responsible for adding base pairs on chromosome ends to maintain telomere length. Telomerase is highly expressed in tissues with constant and rapid cell division, such as cells germline tissue, bone marrow, and linings in the gastrointestinal tract. In contrast, telomerase is usually undetectable or minimally active in mitotic tissues, which results in shorter telomeres with each cell division and leads to the accumulation of senescent cells. From an organism-level perspective, this manifests as aging. A rare autosomal dominant mutation in the gene that codes for the RNA component of telomerase causes premature aging and death, most often from infections related to bone-marrow failure. Because telomerase maintains cell proliferation and division by reducing the erosion of chromosomal ends, mice deficient in TERT, like humans with defective TERT, have shorter telomeres and a shorter life span.

Telomere shortening is observed in every individual with old age. It has been found that people 60-years old or older that have shorter telomeres have three times higher risk of getting heart disease, and longer telomeres are positively correlated to a longer lifespan. Despite this, there are no data to substantiate telomere length as being causative for an increased risk of heart disease or for enabling a longer lifespan. The exact mechanism and correlation of aging and telomere length remains unclear.

Recent studies have shown that expressing exogenous TERT can revert the hyper-aging process caused by a TERT deficiency, suggesting a direct role or balancing act of TERT in the aging process.

Regarding follistatin (FS344), this monomeric secretory protein is expressed in nearly all tissues. In muscle cells, follistatin functions as a negative regulator of myostatin—a myogenesis inhibitory signal protein. Follistatin is thus known to increase skeletal muscle mass, and studies have suggested that it neutralizes the effect of various TGF-β ligands, including myostatin, and activin inhibition complex through a binding to Act RIIB receptor. Follistatin gene knockout mice have retarded growth, skeletal defects, reduced body mass, and die in a few hours after birth, suggesting an important role of follistatin in skeletal and muscle development. Transgenic mice expressing an enhanced level of follistatin show increase muscle mass by 194-327%, suggesting a direct role of follistatin in body mass development. Mice lacking the myostatin gene have reduced number of muscle fibers and smaller muscle fiber size. Myostatin inhibits the expression of myoD and Pax-3, the transcriptional regulators of body mass development, which results in low body mass and small-fiber size. These findings strongly suggest an important role of follistatin in the treatment of muscular dystrophy and other age-related diseases.

The klotho protein is a ubiquitous transmembrane protein that functions to enzymatically hydrolyze steroid β-glucuronides. Klotho plays a role in modulating insulin sensitivity, promoting binding of fibroblast growth factors to their corresponding receptors, regulating calcium homeostasis, minimizing oxidative stress and inflammation, preventing endothelial dysfunction, and promoting myelin integrity and concomitant cognitive function. There are three subtypes of klotho, referred to as α-klotho, β-klotho, and γ-klotho. Each of these subtypes are included in this disclosure and one or more klotho subtypes may be utilized as part of the recombinant CMV payload. Typically, the generic phrase klotho, if not specified otherwise, refers to the α-klotho subtype. Suboptimal levels of klotho protein are associated with degenerative processes such as akin atrophy, osteoporosis, and arteriosclerosis. Low levels of circulating klotho protein are known to be associated with aging. Transgenic mice lacking the α-klotho enzyme develop symptoms of premature aging, whereas transgenic mice that overexpress klotho live longer than wild-type mice.

The Dsup protein is believed to be unique to animals of the phylum Tardigrada, commonly referred to as tardigrades. Tardigrades are ubiquitously present throughout the Earth's biosphere, including in relatively inhospitable locations such as deep sea vents and the Antarctic. Tardigrades are among the most resilient lifeforms known, if not the most resilient. Tardigrades are known to be capable of surviving ionizing radiation at doses hundreds of times higher than lethal levels for humans, low temperatures approaching absolute zero, high temperatures approaching 150° C., pressures about 6 times higher than that found at the deepest levels of the oceans, and even the vacuum of outer space. Dsup functions to protect DNA against damage from ionizing radiation, even when tardigrades are in an active, non-desiccated state. Adding Dsup protein to cultured human cells was shown to reduce DNA damage from X-ray radiation by 40%. While the exact DNA protective mechanism of Dsup is unknown, it is believed to form protective molecular aggregates that associate with nucleosomes in the cell to shield DNA.

PGC-1-α regulates mitochondrial biogenesis and liver gluconeogenesis. Specifically, PGC-1-α is a transcriptional coactivator that functions as an integrator of external stimuli to promote transcription of genes involved in energy metabolism, including promoting the production of new mitochondria. Increased levels of PGC-1-α resulting from aerobic exercise have also been shown to increase autophagy in skeletal muscle.

Oct-4 (octamer-binding transcription factor 4; also known as POU5F1), Sox2 (sex determining region Y-box 2), and KLF4 (Krüppel-like factor 4) are transcription factors that function to activate or deactivate certain expressions related to stem cell differentiation. These proteins play a role in maintaining the pluripotency of certain stem cells, and lower levels promotes stem cell differentiation. These and other similar factors may also be capable of inducing pluripotency, with potential applications for regenerative medicine.

Surprisingly, treatment methods disclosed herein that include administration of recombinant CMV vectors (e.g., murine CMV (MCMV) vectors utilized in the mouse model examples described herein) demonstrated several beneficial effects. In particular, MCMV_{TERT} virus and recombinant MCMV_{FS344} (also referred to herein as MCMV_{FST}) virus demonstrated increased serum expression of TERT and FS344 gene products, respectively, and also increased healthy life span of the subject by up to 40%. Both viral vectors demonstrated increased autophagy and increased number and concentration of mitochondria in skeletal and heart muscle. The disclosed treatments additionally increased physical coordination, increased glucose tolerance, and prevented age-related hair loss—none of which were expected benefits of the disclosed gene therapies. Embodiments of the present disclosure can have multiple applications: preserving the health and muscle of astronauts during low orbit and deep space missions; cost reduction in insurance because of delayed need for care for chronic conditions; possible improvement in type-2 diabetes; and possible protective effects in chronic inflammatory conditions. Many of these are significant risk factors for exaggerated immune reactions during infections with common pathogens. In addition to human health, such therapies could have application in animal health, increasing the lifespan of pets and/or the health of livestock animals, for example. It is also envisioned that embodiments disclosed herein will be utilized in research applications in which the “subject” is a laboratory animal such as a mouse, non-human primate (e.g., rhesus macaque), or other laboratory mammal.

### Recombinant Viral Vectors and Methods of Manufacture and Use

The present disclosure also includes therapeutic compositions containing the recombinant CMV vector and a pharmaceutically acceptable carrier or diluent. The therapeutic composition is useful in the gene therapy and immunotherapy embodiments of the present disclosure, e.g., in a method for transferring genetic information to an animal or human in need of such, which may comprise administering to the subject the composition. Embodiments of the present disclosure accordingly include methods for transferring genetic information.

In another embodiment, methods are provided for generating a recombinant viral vector (e.g., CMV) configured to express a protein, gene product, or expression product following infection or transduction of a cell in vitro or in vivo. Within an in vitro environment, embodiments of the present disclosure provide methods for cloning or replicating a heterologous DNA sequence which may comprise infecting or transfecting a cell in vitro with a recombinant CMV vector disclosed herein and optionally extracting, purifying, and/or isolating the DNA from the cell or progeny virus.

Embodiments of the present disclosure provide, in another aspect, a method for preparing the recombinant CMV vectors disclosed herein, which may comprise inserting the exogenous DNA into a non-essential region of the CMV genome. The method can further include deleting a non-essential region from the CMV genome, preferably prior to inserting the exogenous DNA.

The methods provided herein can include in vivo and/or in vitro recombination. Thus, methods of the present disclosure can include transduction of a cell with CMV DNA into a cell-compatible medium in the presence of donor DNA. The donor DNA may comprise the exogenous DNA flanked by DNA sequences homologous with portions of the CMV genome, whereby the exogenous DNA is introduced into the CMV genome via homologous recombination, and optionally then recovering CMV modified by the in vivo recombination.

The method can also include cleaving CMV DNA to obtain cleaved CMV DNA, ligating the exogenous DNA to the cleaved CMV DNA to obtain hybrid CMV-exogenous DNA, transfecting a cell with the hybrid CMV-exogenous DNA, and optionally then recovering recombinant CMV modified by the presence of the exogenous DNA.

Since in vivo recombination is comprehended, embodiments of the present disclosure accordingly also provide a plasmid, P1-derived artificial chromosome (PAC) and/or bacterial artificial chromosome (BAC) system which comprises donor DNA not naturally occurring in CMV and which encodes a polypeptide foreign to CMV. The donor DNA may be provided within a segment of CMV DNA which would otherwise be co-linear with a non-essential region of the CMV genome such that, after insertion of the donor DNA, DNA from a non-essential region of CMV is replaced by or flanks the donor DNA. Examples of such constructs are described in greater detail below.

The exogenous DNA can be inserted into CMV to generate the recombinant CMV in any orientation which yields stable integration of that DNA, and expression thereof, when desired.

### Example CMV Constructs

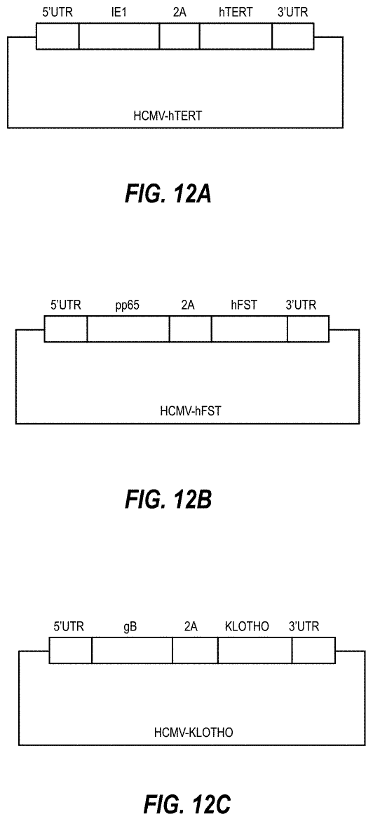

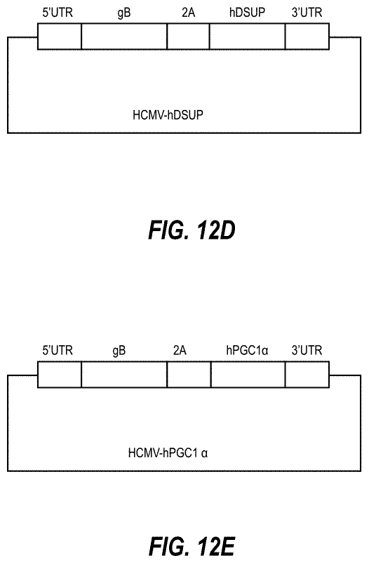

FIGS. 12A-12E illustrate example BAC constructs that may be utilized to generate the CMV vectors disclosed herein. As shown, the constructs include one or more therapeutic genes suitable for administration to human subjects. FIG. 12A shows an example construct that includes hTERT as an exogenous insert. In the illustrated example, the hTERT sequence is fused to the C-terminus (i.e., the portion coding for the C-terminus) of the immediate-early (IE1) gene via a sequence coding for a self-cleaving 2A peptide. The IE1 protein is an early phase protein expressed at high levels during early stages of CMV infection.

FIG. 12B shows an example construct that includes hFST as an exogenous insert. In this example, the hFST sequence is fused to the C-terminus of the pp65 (ppUL83) gene via a sequence coding for a self-cleaving 2A peptide.

FIG. 12C shows an example construct that includes KLOTHO as an exogenous insert. In this example, the KLOTHO sequence is fused to the C-terminus of the gB (envelope glycoprotein B) gene via a sequence coding for a self-cleaving 2A peptide.

FIG. 12D shows an example construct that includes Dsup as an exogenous insert. In this example, the Dsup sequence is fused to the C-terminus of the gB (envelope glycoprotein B) gene via a sequence coding for a self-cleaving 2A peptide.

FIG. 12E shows an example construct that includes PGC1α as an exogenous insert. In this example, the PGC1α sequence is fused to the C-terminus of the gB (envelope glycoprotein B) gene via a sequence coding for a self-cleaving 2A peptide.

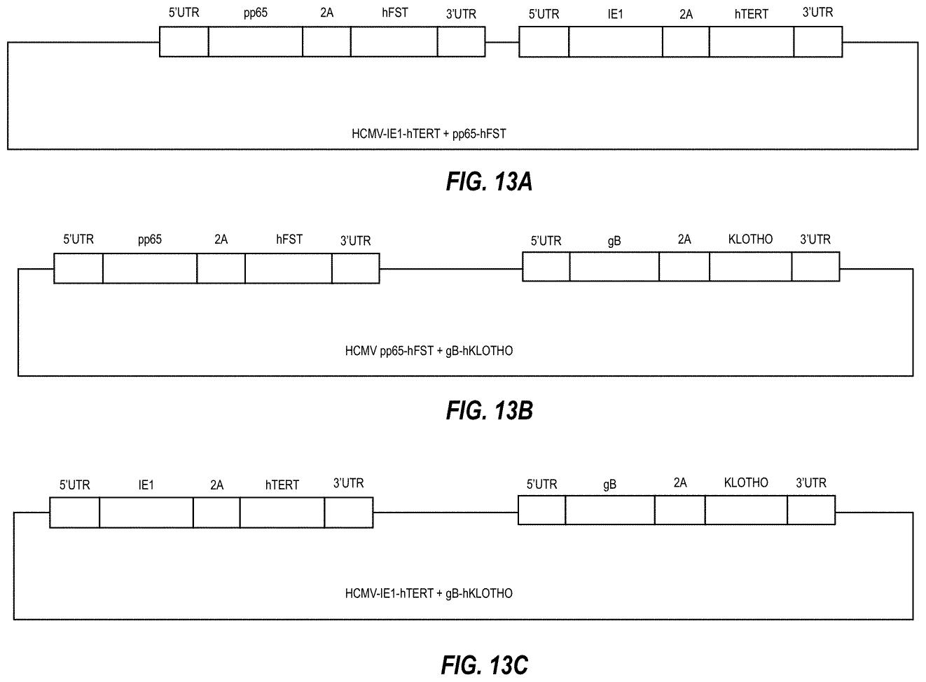

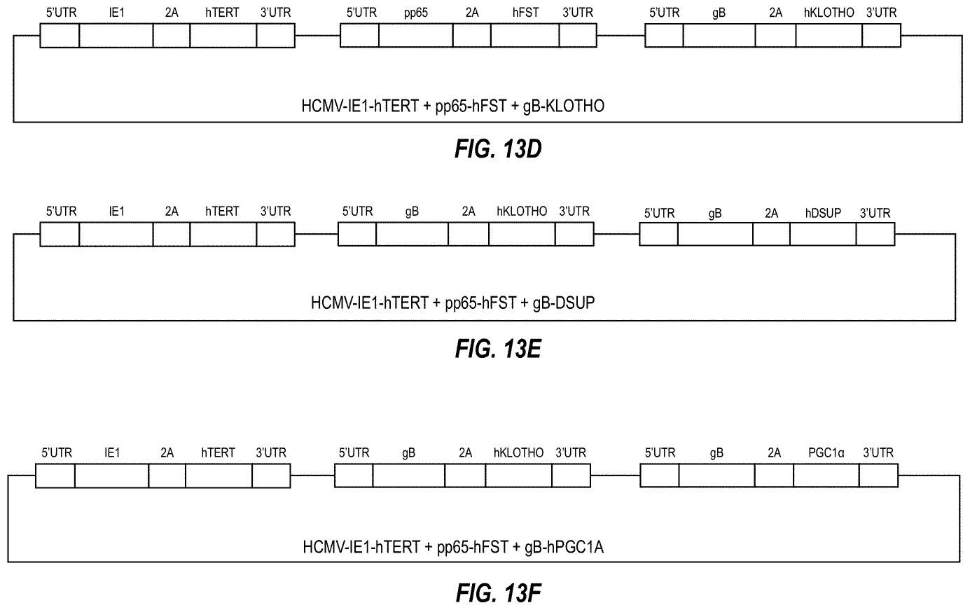

FIGS. 13A-13F illustrate example BAC constructs that each include multiple therapeutic donor genes suitable for administration to human subjects. FIG. 13A shows an example construct that includes both hTERT and hFST as exogenous inserts, wherein the hTERT is joined to the C-terminus of the IE1 gene and the hFST is joined to the C-terminus of the pp65 gene, each via a sequence coding for a self-cleaving 2A peptide.

FIG. 13B shows an example construct that includes both hFST and KLOTHO as exogenous inserts, wherein the hFST is joined to the C-terminus of the pp65 gene and the KLOTHO is joined to the C-terminus of the gB gene, each via a sequence coding for a self-cleaving 2A peptide.

FIG. 13C shows an example construct that includes both hTERT and KLOTHO as exogenous inserts, wherein the hTERT is joined to the C-terminus of the IE1 gene and the KLOTHO is joined to the C-terminus of the gB gene, each via a sequence coding for a self-cleaving 2A peptide.

FIG. 13D shows an example construct that includes hTERT, hFST, and KLOTHO as exogenous inserts, wherein the hTERT is joined to the C-terminus of the IE1 gene, the hFST is joined to the C-terminus of the pp65 gene, and the KLOTHO is jointed to the C-terminus of the gB gene, each via a sequence coding for a self-cleaving 2A peptide.

FIG. 13E shows an example construct that includes hTERT, hFST, and Dsup as exogenous inserts, wherein the hTERT is joined to the C-terminus of the IE1 gene, the hFST is joined to the C-terminus of the pp65 gene, and the Dsup is jointed to the C-terminus of the gB gene, each via a sequence coding for a self-cleaving 2A peptide.

FIG. 13F shows an example construct that includes hTERT, hFST, and PGC1α as exogenous inserts, wherein the hTERT is joined to the C-terminus of the IE1 gene, the hFST is joined to the C-terminus of the pp65 gene, and the PGC1α gene is jointed to the C-terminus of the gB gene, each via a sequence coding for a self-cleaving 2A peptide.

The constructs of this disclosure are not limited to the specific embodiments illustrated in the Figures. For example, other constructs may include one or more donor genes selected from hTERT, hFST, KL, Dsup, PGC1-α, Oct-4, Sox2, KLF4, or any combination thereof. The one or more donor genes may be fused at or near the C-terminus of a CMV gene selected from the IE1 gene, 1E2 gene, pp65 gene, UL21.1 gene, UL21.5, gB gene, TRL4 gene, UL89 gene, US3 gene, R160461 gene, R27080 gene, or other suitable CMV gene or reading frame with sufficient expression. Other CMV genes that may be utilized as fusion sites include genes within the gene families RL11, UL14, UL18, UL25, UL82, UL120, US6, US7, US12, and US22. Additional or alternative CMV genes may be utilized as fusion sites for the one or more donor genes. Preferable fusion sites are those that are expressed at relatively high levels to in turn enable high relative expression of the fused gene product. Presently preferred CMV genes to utilized as fusion sites for the one or more donor genes include the IE1 gene, pp65 gene, and gB gene. The one or more donor genes may be fused to their respective CMV genes via a sequence coding for a 2A self-cleaving peptide selected from T2A, P2A, E2A, or F2A, for example.

In some embodiments, a construct includes two donor genes. For example, a construct may include: hTERT and hFST; hTERT and KLOTHO; hTERT and Dsup; hTERT and PGC1-α; hTERT and Oct-4; hTERT and Sox2; hTERT and KLF4; hFST and KLOTHO; hFST and Dsup; hFST and PGC1-α; hFST and Oct-4; hFST and Sox2; hFST and KFL4; KLOTHO and Dsup; KLOTHO and PGC1-α; KLOTHO and Oct-4; KLOTHO and Sox2; KLOTHO and KLF4; Dsup and PGC1-α; Dsup and Oct-4; Dsup and Sox2; Dsup and KLF4; PGC1-α and Oct-4; PGC1-α and Sox2; PGC1-α and KLF4; Oct-4 and Sox2; Oct-4 and KLF4; or Sox2 and KLF4.

In some embodiments, a construct includes three donor genes. For example, a construct may include: hTERT, hFST, and KLOTHO; h IERT, hFST, and Dsup; hTERT, hFST, and PGC1-α; hTERT, hFST, and Oct-4; hTERT, hFST, and Sox2; hTERT, hFST, and KLF4; hTERT, KLOTHO, and Dsup; hTERT, KLOTHO, and PGC1-α; hTERT, KLOTHO, and Oct-4; hTERT, KLOTHO, and Sox2; hTERT, KLOTHO, and KLF4; hTERT, Dsup, and PGC1-α; hTERT, Dsup, and Oct-4; hTERT, Dsup, and Sox2; hTERT, Dsup, and KLF4; h IERT, PGC1-α, and Oct-4; hTERT, PGC1-α, and Sox2; hTERT, PGC1-α, and KLF4; h IERT, Oct-4, and Sox2; hTERT, Oct-4, and KLF4; hTERT, Sox2, and KLF4; hFST, KLOTHO, and Dsup; hFST, KLOTHO, and PGC1-α; hFST, KLOTHO, and Oct-4; hFST, KLOTHO, and Sox2; hFST, KLOTHO, and KLF4; hFST, Dsup, and PGC1-α; hFST, Dsup, and Oct-4; hFST, Dsup, and Sox2; hFST, Dsup, and KLF4; hFST, PGC1-α, and Oct-4; hFST, PGC1-α, and Sox2; hFST, PGC1-α, and KLF4; hFST, Oct-4, and Sox2; hFST, Oct-4, and KLF4; hFST, Sox2, and KLF4; KLOTHO, Dsup, and PGC1-α; KLOTHO, Dsup, and Oct-4; KLOTHO, Dsup, and Sox2; KLOTHO, Dsup, and KLF4; KLOTHO, PGC1-α, and Oct-4; KLOTHO, PGC1-α, and Sox2; KLOTHO, PGC1-α, and KLF4; KLOTHO, Oct-4, and Sox2; KLOTHO, Oct-4, and KLF4; KLOTHO, Sox2, and KLF4; Dsup, PGC1-α, and Oct-4; Dsup, PGC1-α, and Sox2; Dsup, PGC1-α, and KLF4; Dsup, Oct-4, and Sox2; Dsup, Oct-4, and KLF4; Dsup, Sox2, and KLF4; PGC1-α, Oct-4, and Sox2; PGC1-α, Oct-4, and KLF4; PGC1-α, Sox2, and KLF4; or Oct-4, Sox2, and KLF4.

Other embodiments may include more than three donor genes, in any combination. For example, a CMV construct may include more than three of hTERT, hFST, KL, Dsup, PGC1-α, Oct-4, Sox2, and KLF4, in any combination.

In each of the foregoing, the CMV genes utilized as fusion sites may be selected from any of the CMV fusion sites disclosed herein; preferably the fusion sites are selected from the IE1 gene, pp65 gene, and gB gene.

In construct embodiments that include more than one donor gene, each donor gene may be positioned in a separate open reading frame. Alternatively, one or more donor genes may share the same open reading frame. For example, each donor gene may be fused to a separate CMV gene, or alternatively, one or more donor genes may be fused together to form a donor gene fusion product which is then fused to an appropriate CMV gene. Such fused donor genes may be fused (to each other and/or to the CMV gene) via a sequence coding for a 2A self-cleaving peptide, as with other embodiments described herein. Expression of the donor genes may be induced by the same promoter. Alternatively, the donor genes can be independently associated with separate and/or unique promoters that allow individual or separate expression of each donor gene.

Moreover, although BAC constructs represent presently preferred construct embodiments, other construct embodiments may utilize alternative backbones such as P1-derived artificial chromosomes (PACs) and/or suitable plasmids. Where plasmids are utilized, due to the more stringent size constraints, transduction methods may utilize separate, different plasmids that together provide the desired combination of donor genes.

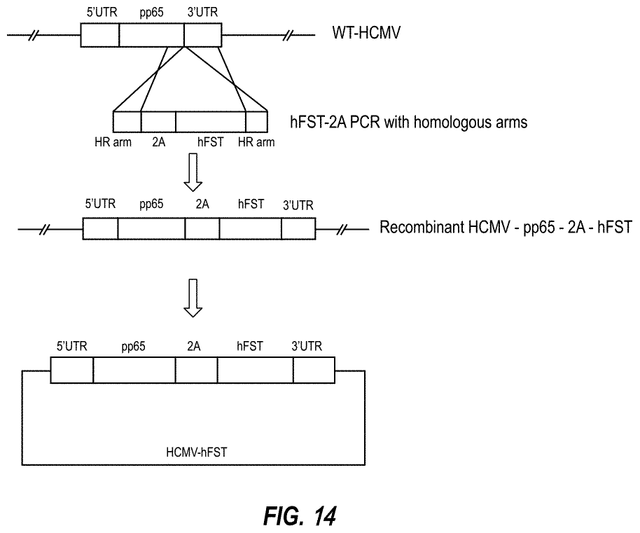

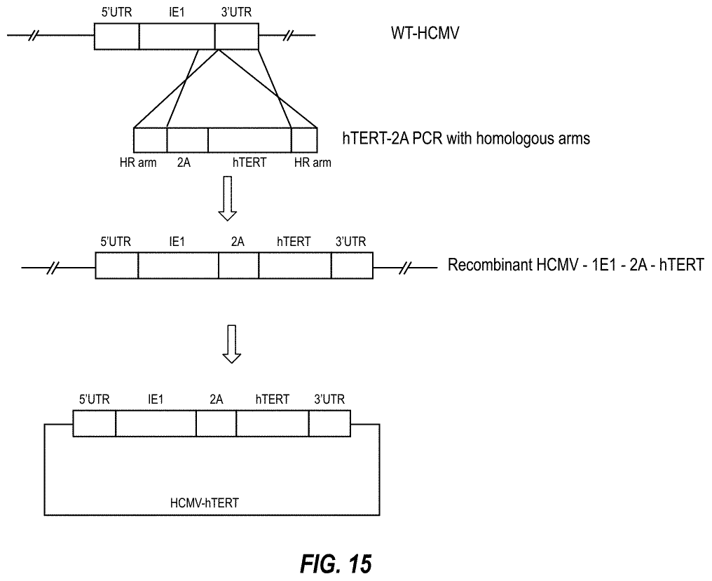

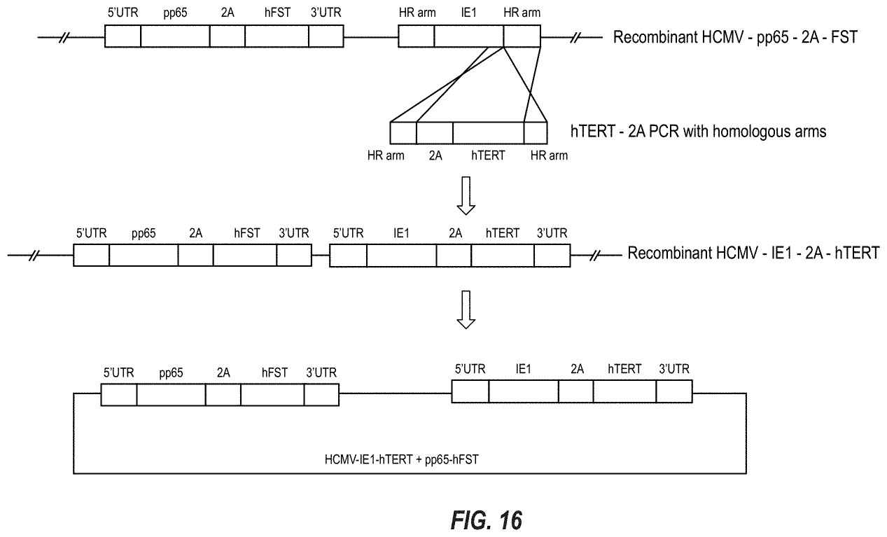

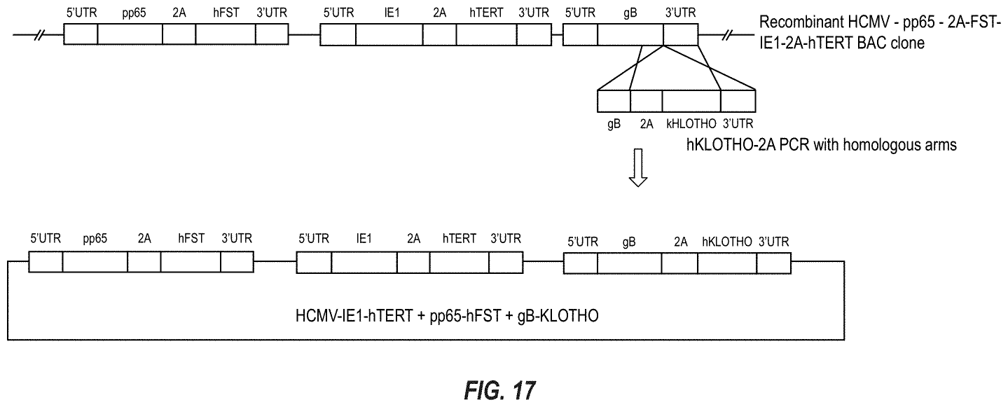

FIGS. 14-17 illustrate methods for manufacturing a CMV-hFST recombinant BAC, CMV-hTERT recombinant BAC, CMV-hFST+hTERT recombinant BAC, and a CMV-hFST+hTERT+KL recombinant BAC, respectively, via homologous recombination. As shown in these figures, the insert is provided with homologous arms (HR arms) on both upstream and downstream ends. The HR arms have substantial identity to the target site in the CMV genome. For example, the upstream HR arm may have substantial identity to a downstream section (i.e., closer to the C-terminus) of the CMV gene targeted for fusion, whereas the downstream HR arm may have substantial identity to the 3′ untranslated region disposed immediately downstream from the C-terminus of the target CMV gene. In this manner, the insert may be inserted into position between the C-terminus of the target CMV gene and the 3′ untranslated region through homologous recombination.

The HR arms may be any length that provides sufficient homologous recombination and integration of the insert into the BAC backbone. In some embodiments, the homologous arms are at least about 25 base pairs in length, or at least about 30 base pairs in length, or at least about 35 base pairs in length, at least about 40 base pairs in length, at least about 45 base pairs in length, and may be up to about 50 base pairs in length, or up to about 55 base pairs in length, or up to about 60 base pairs in length, or up to about 65 base pairs in length, or up to about 70 base pairs in length, or up to about 75 base pairs in length, or up to about 80 base pairs in length, or up to about 90 base pairs in length, or up to about 100 base pairs in length. The HR arms may have a length within a range with endpoints defined by any two of the foregoing values. Effective results have been achieved using HR arms of between about 50 base pairs and about 80 base pairs. Smaller sizes may result in reduced recombination efficiency, while sizes that are too large increase the complexity of the method and are more likely to introduce unwanted side reactions.

### Additional Recombinant Viral Vector Details

The exogenous DNA in the recombinant CMV viruses or vectors described herein can include a promoter. The promoter can be from a herpes virus. For instance, the promoter can be a cytomegalovirus (CMV) promoter, such as a human CMV (HCMV) or murine CMV (MCMV) promoter. The promoter can also be a non-viral promoter such as the EFla promoter. The promoter may be a truncated transcriptionally active promoter which may comprise a region transactivated with a transactivating protein provided by the virus and the minimal promoter region of the full-length promoter from which the truncated transcriptionally active promoter is derived. For purposes of this specification, a “promoter” is composed of an association of DNA sequences corresponding to the minimal promoter and upstream regulatory sequences; a “minimal promoter” is composed of the CAP site plus TATA box (minimum sequences for basic level of transcription; unregulated level of transcription); and “upstream regulatory sequences” are composed of the upstream element(s) and enhancer sequence(s). Further, the term “truncated” indicates that the full-length promoter is not completely present (i.e., that some portion of the full-length promoter has been removed), and the truncated promoter can be derived from a herpesvirus such as MCMV or HCMV (e.g., HCMV-IE or MCMV-IE). Exemplary truncated promoters can be up to a 40% and even up to a 90% reduction in size, from a full-length promoter, based upon base pairs. The promoter can also be a modified non-viral promoter.

Embodiments of the present disclosure also provide an expression cassette for insertion into a recombinant virus or plasmid which may include the truncated transcriptionally active promoter. The expression cassette can further include a functional truncated polyadenylation signal, such as an SV40 polyadenylation signal which is truncated, yet functional. Considering that nature provided a larger signal, it is indeed surprising that a truncated polyadenylation signal is functional; and a truncated polyadenylation signal addresses the insert size limit problems of recombinant viruses such as CMV. The expression cassette can also include exogenous or heterologous DNA with respect to the virus or system into which it is inserted, and that DNA can be exogenous or heterologous DNA as described herein. This DNA can be suitably positioned and operably linked to the promoter for expression. As to HCMV promoters, reference is made to U.S. Pat. Nos. 5,168,062 and 5,385,839, each of which is incorporated herein.

The heterologous or exogenous DNA in exemplary disclosed recombinants preferably encode an expression product, such as a therapeutic gene (e.g., TERT and/or FS344, including human and/or mouse versions thereof, or other version thereof appropriate for the target subject) and/or a fusion protein (e.g., fused with a reporter, such as luciferase, or with an N- or C-terminal epitope tag, such as FLAG, 6×-His, or other epitope tag known to those having skill in the art). With respect to these terms, reference is made to the following discussion, and generally to Kendrew, THE ENCYCLOPEDIA OF MOLECULAR BIOLOGY (Blackwell Science Ltd 1995) and Sambrook, Fritsch, Maniatis, Molecular Cloning, A LABORATORY MANUAL (2d Edition, Cold Spring Harbor Laboratory Press, 1989).

As to size of the DNA incorporated to form recombinant virus/vectors: the skilled artisan can maximize the size of the protein encoded by the DNA sequence to be inserted into the selected viral vector (keeping in mind the packaging limitations of the vector). To minimize the DNA inserted while maximizing or matching the native size of the protein(s) expressed, the DNA sequence(s) can exclude introns (regions of a gene that are transcribed but which are subsequently excised from the primary RNA transcript prior to translation).

With respect to expression of fusion proteins disclosed herein, reference is made to Sambrook, Fritsch, Maniatis, Molecular Cloning, A LABORATORY MANUAL (2d Edition, Cold Spring Harbor Laboratory Press, 1989) (especially Volume 3), and Kendrew, THE ENCYCLOPEDIA OF MOLECULAR BIOLOGY (Blackwell Science Ltd 1995. The teachings of Sambrook et al., can be suitably modified, without undue experimentation, from this disclosure, for the skilled artisan to generate recombinants expressing fusion proteins. With regard to gene therapy, reference is made to U.S. Pat. No. 5,252,479, which is incorporated herein by this reference, together with the documents cited therein and on its face.

It should be understood that the proteins and the nucleic acids encoding them may differ from the exact sequences illustrated and described herein. Thus, the invention contemplates deletions, additions, truncations, and substitutions to the sequences shown, so long as the sequences function in accordance with the methods of the invention. In this regard, substitutions will generally be conservative in nature (i.e., those substitutions that take place within a family of amino acids). For example, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. It is reasonably predictable that an isolated replacement of a leucine with an isoleucine or valine, or vice versa; an aspartate with a glutamate or vice versa; a threonine with a serine or vice versa; or a similar conservative replacement of an amino acid with a structurally related amino acid, will typically not have a major effect on the biological activity. Proteins having substantially the same amino acid sequence as the sequences illustrated and described but possessing minor amino acid substitutions that do not substantially affect the activity or function of the protein are, therefore, within the scope of the invention.

In some embodiments, the therapeutic gene includes a recombinant nucleotide sequence. For example, in one embodiment the nucleotide sequences may be codon optimized, for example the codons may be optimized for human use. As regards codon optimization, the nucleic acid molecules of the invention have a nucleotide sequence that encodes the therapeutic proteins of the invention (i.e., telomerase and/or follistatin) and can be designed to employ codons that are used in the genes of the subject in which the protein is to be produced. Many viruses use a large number of rare codons and, by altering these codons to correspond to codons commonly used in the desired subject, enhanced expression of the recombinant genes can be achieved. In a preferred embodiment, the codons used are “humanized” codons, i.e., the codons are those that appear frequently in highly expressed human genes (see Andre et al., J. Virol. 72:1497-1503, 1998) instead of those codons that are frequently used by or encoded by the vector/virus. Such codon usage provides for efficient expression of the transgenic viral proteins in human cells. Any suitable method of codon optimization may be used. Such methods, and the selection of such methods, are well known to those of skill in the art, and the nucleotide sequences of the inventive vectors described herein can readily be codon optimized in light of the additional teachings provided by this disclosure.

The invention further encompasses nucleotide sequences encoding functionally equivalent variants and derivatives of the CMV vectors disclosed herein. These functionally equivalent variants, derivatives, and fragments display the ability to retain functional activity. For instance, changes in a DNA sequence that do not change the encoded amino acid sequence, as well as those that result in conservative substitutions of amino acid residues, one or a few amino acid deletions or additions, and substitution of amino acid residues by amino acid analogs are those which will not significantly affect properties of the encoded polypeptide. Conservative amino acid substitutions include glycine/alanine; valine/isoleucine/leucine; asparagine/glutamine; aspartic acid/glutamic acid; serine/threonine/methionine; lysine/arginine; and phenylalanine/tyrosine/tryptophan. In some embodiments, the nucleotides have at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% homology or identity to the natural form of the polypeptide of interest.

For the purposes of the present invention, sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993; 90: 5873-5877. Another example of a mathematical algorithm used for comparison of sequences is the algorithm of Myers & Miller, CABIOS 1988; 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988; 85: 2444-2448. Advantageous for use according to the present invention is the WU-BLAST (Washington University BLAST) version 2.0 software. WU-BLAST version 2.0 executable programs for several UNIX platforms can be downloaded from ftp://blast.wust1.edu/blast/executables. This program is based on WU-BLAST version 1.4, which in turn is based on the public domain NCBI-BLAST version 1. (Altschul & Gish, 1996, Local alignment statistics, Doolittle ed., Methods in Enzymology 266: 460-480; Altschul et al., Journal of Molecular Biology 1990; 215: 403-410; Gish & States, 1993; Nature Genetics 3: 266-272; Karlin & Altschul, 1993; Proc. Natl. Acad. Sci. USA 90: 5873-5877).

The various recombinant nucleotide sequences and recombinant vectors can be made using standard recombinant DNA and cloning techniques, such as those disclosed in “Molecular Cloning: A Laboratory Manual”, second edition (Sambrook et al. 1989). However, it should generally be appreciated that the vectors used in accordance with the embodiments of the present disclosure are typically chosen such that they contain a suitable gene regulatory region, such as a promoter or enhancer, to allow for the desired level of expression of the encoded therapeutic gene. Thus, one skilled in the art can create recombinants expressing a therapeutic gene of interest and use the recombinants from this disclosure and the knowledge in the art, without undue experimentation. Moreover, from the disclosure herein and the knowledge in the art, no undue experimentation is required for the skilled artisan to construct a recombinant virus/vector that expresses a therapeutic gene of interest or for the skilled artisan to use such a recombinant virus/vector.

Embodiments of the present disclosure enable various treatment methods. For example, a treatment method can include a longitudinal treatment method where a plurality of therapeutically effective dosages are provided to a patient over a period of time. In some instances, the period of time is as long as 6-12 months or more, with dosages being administered to the patient annually, semi-annually, every other month, every month, every three weeks, every other week, every week, or more regularly, for example. In an exemplary treatment method, a therapeutically effective dosage is administered to the patient every month for 8 months. In some embodiments, the patient is a middle-aged or elderly patient. For example, the patient can be a human patient that is 30 years or older, 35 years or older, 40 years or older, 45 years or older, 50 years or older, 55 years or older, 60 years or older, 65 years or older, 70 years or older, or older (or can be any age falling within a range formed by the foregoing ages). The patient can also be an animal patient having an analogously middle-age or elderly age.

### Pharmaceutical Compositions

While it is possible for the compounds described herein to be administered alone, it may be preferable to formulate the compounds as pharmaceutical compositions (e.g., formulations). As such, in yet another aspect, pharmaceutical compositions useful in the methods and uses of the disclosed embodiments are provided. A pharmaceutical composition is any composition that may be administered in vitro or in vivo or both to a subject to treat, prevent, or ameliorate a condition or may otherwise be administered prophylactically to improve or maintain the health of the subject. In a preferred embodiment, a pharmaceutical composition may be administered in vivo. A subject may include one or more cells or tissues, or organisms. In some exemplary embodiments, the subject is an animal. In some embodiments, the animal is a mammal. The mammal may be a human, mouse, or primate in some embodiments. A mammal includes any mammal, such as by way of non-limiting example, cattle, pigs, sheep, goats, horses, camels, buffalo, primates, cats, dogs, rats, mice, and humans.

The compositions of the invention may be injectable suspensions, solutions, sprays, lyophilized powders, syrups, elixirs, and the like. Any suitable form of composition may be used. To prepare such a composition, a nucleic acid or vector of the invention, having the desired degree of purity, is mixed with one or more pharmaceutically acceptable carriers and/or excipients.

As used herein the terms “pharmaceutically acceptable” and “physiologically acceptable” mean a biologically compatible formulation, gaseous, liquid, or solid, or mixture thereof, which is suitable for one or more routes of administration, in vivo delivery, or contact. A formulation is compatible in that it does not destroy activity of the engineered viral vector or proteins made thereby or induce adverse side effects that outweigh any prophylactic or therapeutic effect or benefit.

In an embodiment, the pharmaceutical compositions may be formulated with pharmaceutically acceptable excipients such as carriers, solvents, stabilizers, adjuvants, diluents, etc., depending upon the particular mode of administration and dosage form. The pharmaceutical compositions should generally be formulated to achieve a physiologically compatible pH and may range from a pH of about 3 to a pH of about 11, preferably about pH 3 to about pH 7, depending on the formulation and route of administration. In alternative embodiments, it may be preferred that the pH is adjusted to a range from about pH 5 to about pH 8. More particularly, the pharmaceutical compositions may comprise a therapeutically or prophylactically effective amount of at least one compound as described herein, together with one or more pharmaceutically acceptable excipients. Optionally, the pharmaceutical compositions may comprise a combination of the compounds described herein or may include a second recombinant vector, or second protein encoded thereby, that is useful in the treatment or prevention of aging or aging-related phenomena, as discussed herein.

Formulations, for example, for parenteral or oral administration, are most typically solids, liquid solutions, emulsions, or suspensions, while inhalable formulations for intranasal or pulmonary administration are generally liquids or powders. An exemplary pharmaceutical composition may be formulated as a lyophilized solid that is reconstituted with a physiologically compatible solvent or carrier prior to administration. Other suitable carriers or diluents can be water or a buffered saline, with or without a preservative. The recombinant vector may be lyophilized for resuspension at the time of administration or can be in solution.

Acceptable carriers, excipients, or stabilizers are nontoxic to recipients at the dosages and concentrations employed, and include, but are not limited to, water, saline, phosphate buffered saline, dextrose, glycerol, ethanol, or combinations thereof, buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN® PLURONICS® or polyethylene glycol (PEG).

Pharmaceutically acceptable excipients are determined in part by the particular composition being administered, as well as by the particular method used to administer the composition. Accordingly, there exists a wide variety of suitable formulations of pharmaceutical compositions (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants such as ascorbic acid; chelating agents such as EDTA; carbohydrates such as dextrin, hydroxyalkylcellulose, hydroxyalkylmethylcellulose, stearic acid; liquids such as oils, water, saline, glycerol, and ethanol; wetting or emulsifying agents; pH buffering substances; and the like. Liposomes are also included within the definition of pharmaceutically acceptable excipients.

For example, pharmaceutically acceptable excipients particularly suitable for use in conjunction with tablets include, for example, inert diluents, such as celluloses, calcium or sodium carbonate, lactose, calcium, or sodium phosphate; disintegrating agents, such as cross-linked povidone, maize starch, or alginic acid; binding agents, such as povidone, starch, gelatin, or acacia; and lubricating agents, such as magnesium stearate, stearic acid, or talc. Pharmaceutical compositions may also be formulated as dispersible powders and granules suitable for preparation of a suspension by the addition of suitable excipients.

As another example, pharmaceutical compositions may be formulated as suspensions comprising a recombinant vector/virus disclosed herein in admixture with at least one pharmaceutically acceptable excipient suitable for the manufacture of a suspension. Excipients suitable for use in connection with suspensions include suspending agents, such as sodium carboxymethylcellulose, methylcellulose, hydroxypropyl methylcellulose, sodium alginate, polyvinylpyrrolidone, gum tragacanth, gum acacia, dispersing, or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); polysaccharides and polysaccharide-like compounds (e.g., dextran sulfate); glycoaminoglycans and glycosaminoglycan-like compounds (e.g., hyaluronic acid); and thickening agents, such as carbomer, beeswax, hard paraffin, or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

The immunogenic compositions can be designed to introduce the viral vectors to a desired site of action and release it at an appropriate and controllable rate. For example, controlled release preparations can be produced by the use of polymers to complex or absorb the immunogen and/or immunogenic composition. A controlled-release formulation can be prepared using appropriate macromolecules (for example, polyesters, polyamino acids, polyvinyl, pyrrolidone, ethylenevinylacetate, methylcellulose, carboxymethylcellulose, or protamine sulfate) known to provide the desired controlled release characteristics or release profile. Another possible method to control the duration of action by a controlled-release preparation is to incorporate the active ingredients into particles of a polymeric material such as, for example, polyesters, polyamino acids, hydrogels, polylactic acid, polyglycolic acid, copolymers of these acids, or ethylene vinylacetate copolymers.

Alternatively, instead of incorporating these active ingredients into polymeric particles, it is possible to entrap these materials into microcapsules. Microencapsulation has been applied to the injection of microencapsulated pharmaceuticals to give a controlled release. A number of factors contribute to the selection of a particular polymer for microencapsulation. The reproducibility of polymer synthesis and the microencapsulation process, the cost of the microencapsulation materials and process, the toxicological profile, the requirements for variable release kinetics and the physicochemical compatibility of the polymer and the viral vectors are all factors that must be considered. Examples of useful polymers are polycarbonates, polyesters, polyurethanes, polyorthoesters, and polyamides, particularly those that are biodegradable.

Microcapsules can be prepared, for example, by coacervation techniques or by interfacial polymerization, for example, hydroxymethylcellulose or gelatin-microcapsule and poly-(methylmethacrylate) microcapsule, respectively, in colloidal drug delivery systems (for example, liposomes, albumin microspheres, microemulsions, nanoparticles and nanocapsules) or in macroemulsions. Such techniques are disclosed in New Trends and Developments in Vaccines, Voller et al. (eds.), University Park Press, Baltimore, Md., 1978 and Remington's Pharmaceutical Sciences, 16th edition.

A frequent choice of a carrier for pharmaceuticals is poly (d,l-lactide-co-glycolide) (PLGA). This is a biodegradable polyester that has a long history of medical use in erodible sutures, bone plates, and other temporary prostheses where it has not exhibited any toxicity. A wide variety of pharmaceuticals, including peptides and antigens, have been formulated into PLGA microcapsules. A body of data has accumulated on the adaption of PLGA for the controlled release of compounds, for example, as reviewed by Eldridge, J. H., et al., Current Topics in Microbiology and Immunology. 1989, 146:59-66. The entrapment of compounds in PLGA microspheres of 1 to 10 microns in diameter has been shown to have a remarkable adjuvant effect when administered orally. The PLGA microencapsulation process uses a phase separation of a water-in-oil emulsion. The compound of interest is prepared as an aqueous solution and the PLGA is dissolved in suitable organic solvents such as methylene chloride and ethyl acetate. These two immiscible solutions are co-emulsified by high-speed stirring. A non-solvent for the polymer is then added, causing precipitation of the polymer around the aqueous droplets to form embryonic microcapsules. The microcapsules are collected and stabilized with one of an assortment of agents (polyvinyl alcohol (PVA), gelatin, alginates, polyvinylpyrrolidone (PVP), methyl cellulose), and the solvent removed by either drying in vacuo or by solvent extraction.

The pharmaceutical compositions may also be in the form of oil-in water emulsions. The oil-in-water emulsion can be based, for example, on light liquid paraffin oil (European Pharmacopea type); isoprenoid oil such as squalane, squalene, EICOSANE™ or tetratetracontane; oil resulting from the oligomerization of alkene(s), e.g., isobutene or decene; esters of acids or of alcohols containing a linear alkyl group, such as plant oils, ethyl oleate, propylene glycol di(caprylate/caprate), glyceryl tri(caprylate/caprate) or propylene glycol dioleate; esters of branched fatty acids or alcohols, e.g., isostearic acid esters. The oil advantageously is used in combination with emulsifiers to form the emulsion. The emulsifiers can be nonionic surfactants, such as esters of sorbitan, mannide (e.g., anhydromannitol oleate), glycerol, polyglycerol, propylene glycol, and oleic, isostearic, ricinoleic, or hydroxystearic acid, which are optionally ethoxylated, and polyoxypropylene-polyoxyethylene copolymer blocks, such as the Pluronic® products, e.g., L121. The adjuvant can be a mixture of emulsifier(s), micelle-forming agent, and oil such as that which is commercially available under the name Provax® (IDEC Pharmaceuticals, San Diego, Calif.). The emulsion may also contain sweetening and flavoring agents. Syrups and elixirs may be formulated with sweetening agents, such as glycerol, sorbitol, or sucrose. Such formulations may also contain a demulcent, a preservative, a flavoring, or a coloring agent.

Additionally, the pharmaceutical compositions may be in the form of a sterile injectable preparation, such as a sterile injectable aqueous emulsion or oleaginous suspension. This emulsion or suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,2-propane-diol.

The sterile injectable preparation may also be prepared as a lyophilized powder. Among the acceptable vehicles and solvents that may be employed are water, Ringer's solution, and an isotonic sodium chloride solution. In addition, sterile fixed oils may be employed as a solvent or suspending medium. For this purpose, any bland fixed oil may be employed including synthetic mono- or diglycerides. In addition, fatty acids such as oleic acid may be used in the preparation of injectables.

In some embodiments, cyclodextrins may be added as aqueous solubility enhancers. Preferred cyclodextrins include hydroxypropyl, hydroxyethyl, glucosyl, maltosyl and maltotriosyl derivatives of α-, β-, and γ-cyclodextrin. An exemplary cyclodextrin solubility enhancer is hydroxypropyl-o-cyclodextrin (BPBC), which may be added to any of the above-described compositions to further improve the aqueous solubility characteristics of the compounds of the embodiments. In one embodiment, the composition comprises about 0.1% to about 20% hydroxypropyl-o-cyclodextrin, more preferably about 1% to about 15% hydroxypropyl-o-cyclodextrin, and even more preferably from about 2.5% to about 10% hydroxypropyl-o-cyclodextrin. The amount of solubility enhancer employed will depend on the amount of the compound of the embodiments in the composition.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, but are not limited to, mineral salts (e.g., AlK(SO₄)₂, AlNa(SO₄)₂, AlNH(SO₄)₂, silica, alum, Al(OH)₃, Ca₃(PO₄)₂, kaolin, or carbon), polynucleotides with or without immune stimulating complexes (ISCOMs) (e.g., CpG oligonucleotides, such as those described in Chuang, T. H. et al., (2002) J. Leuk. Biol. 71(3): 538-44; Ahmad-Nejad, P. et al. (2002) Eur. J. Immunol. 32(7): 1958-68; poly IC or poly AU acids, polyarginine with or without CpG (also known in the art as IC31; see Schellack, C. et al. (2003) Proceedings of the 34th Annual Meeting of the German Society of Immunology; Lingnau, K. et al. (2002) Vaccine 20(29-30): 3498-508), JuvaVax (U.S. Pat. No. 6,693,086), certain natural substances (e.g., wax D-form Mycobacterium tuberculosis, substances found in Cornyebacterium parvum, Bordetella pertussis, or members of the genus Brucella), flagellin (Toll-like receptor 5 ligand; see McSorley, S. J. et al. (2002) J. Immunol. 169(7): 3914-9), saponins such as Q521, Q517, and QS7 (U.S. Pat. Nos. 5,057,540; 5,650,398; 6,524,584; 6,645,495), monophosphoryl lipid A, in particular, 3-de-O-acylated monophosphoryl lipid A (3D-MPL), imiquimod (also known in the art as IQM and commercially available as Aldara®); U.S. Pat. Nos. 4,689,338; 5,238,944; Zuber, A. K. et al. (2004) 22(13-14): 1791-8), and the CCR5 inhibitor CMPD167 (see Veazey, R. S. et al. (2003) J. Exp. Med. 198: 1551-1562). Aluminum hydroxide or phosphate(alum) are commonly used at 0.05 to 0.1% solution in phosphate buffered saline. Other adjuvants that can be used include cholera toxin, especially CTA1-DD/ISCOMs (see Mowat, A. M. et al. (2001) J. Immunol. 167(6): 3398-405); polyphosphazenes (Allcock, H. R. (1998) App. Organometallic Chem. 12(10-11): 659-666; Payne, L. G. et al. (1995) Pharm. Biotechnol. 6: 473-93); cytokines such as, but not limited to, IL-2, IL-4, GM-CSF, IL-12, IL-15 IGF-1, IFN-α, IFN-β, and IFN-γ (Boyer et al., (2002) J. Liposome Res. 121:137-142; WO01/095919); immunoregulatory proteins such as CD4OL (ADX40; see, for example, WO03/063899), and the CD1a ligand of natural killer cells (also known as CRONY or a-galactosyl ceramide; see Green, T. D. et al., (2003) J. Virol. 77(3): 2046-2055); immunostimulatory fusion proteins such as IL-2 fused to the Fc fragment of immunoglobulins (Barouch et al., Science 290:486-492, 2000); and co-stimulatory molecules B7.1 and B7.2 (Boyer)—all of which can be administered either as proteins or in the form of DNA, in the same viral vectors as those encoding the therapeutic gene(s) of the embodiments disclosed herein or on separate expression vectors.

### Exemplary Dosages and Treatment Regimens

Pharmaceutical compositions disclosed herein contain a total amount of the active ingredient(s) sufficient to achieve an intended therapeutic effect. The pharmaceutical compositions may, for convenience, be prepared or provided as a unit dosage form. Preparation techniques include bringing into association the active ingredient (e.g., the recombinant virus/vectors) and pharmaceutical carrier(s) and/or excipient(s). In general, pharmaceutical compositions are prepared by uniformly and intimately associating the active ingredient with liquid carriers or finely divided solid carriers or both, and then, if necessary, shaping the product. For example, a tablet may be made by compression or molding. Compressed tablets may be prepared by compressing, in a suitable machine, an active ingredient in a free-flowing form such as a powder or granules, optionally mixed with a binder, lubricant, inert diluent, preservative, surface-active or dispersing agent. Molded tablets may be produced by molding, in a suitable apparatus, a mixture of powdered compound moistened with an inert liquid diluent. The tablets may optionally be coated or scored and may be formulated so as to provide a slow or controlled release of the active ingredient therein.

Compounds including disclosed pharmaceutical compositions can be packaged in unit dosage forms for ease of administration and uniformity of dosage. A “unit dosage form” as used herein refers to a physically discrete unit suited as unitary dosages for the subject to be treated; each unit containing a predetermined quantity of compound optionally in association with a pharmaceutical carrier (e.g., excipient, diluent, vehicle or filling agent) which, when administered in one or more doses, is calculated to produce a desired effect (e.g., prophylactic or therapeutic effect or benefit). Unit dosage forms can contain a weekly or monthly dose, or an appropriate fraction thereof, of an administered compound. Unit dosage forms also include, for example, capsules, troches, cachets, lozenges, tablets, ampules, and vials, which may include a composition in a freeze-dried or lyophilized state; a sterile liquid carrier, for example, can be added prior to administration or delivery in vivo. Unit dosage forms additionally include, for example, ampules and vials with liquid compositions disposed therein. The individual unit dosage forms can be included in multi-dose kits or containers. Pharmaceutical formulations can be packaged in single or multiple unit dosage forms for ease of administration and uniformity of dosage.

The compositions disclosed herein can be administered in accordance with the methods at any frequency and as a single bolus or multiple doses, for as long as appropriate. Exemplary frequencies are typically from 1-5 times, 1-3 times, 2-times, or once monthly. Timing of contact and administration ex vivo or in vivo can be dictated by the infection or pathogenesis of the viral vector used or by the concentration of the therapeutic protein in the patient (e.g., in serum or within specified organ tissue). In some embodiments, the compounds will be administered for a period of continuous therapy, for example for a week or more, or for months or years. Long-acting pharmaceutical compositions may be administered twice a week, every 3 to 4 days, every week, or once a month depending on half-life and clearance rate of the particular formulation. For example, in an embodiment, a pharmaceutical composition contains an amount of a compound as described herein that is selected for administration to a patient once a month for 6-12 months.

Doses may vary depending upon whether the treatment is therapeutic or prophylactic, the onset, progression, severity, frequency, duration, probability of or susceptibility of the age-related symptom, the type pathogenesis to which treatment is directed, clinical endpoint desired, previous, simultaneous or subsequent treatments, general health, age, gender or race of the subject, bioavailability, potential adverse systemic, regional or local side effects, the presence of other disorders or diseases in the subject, and other factors that will be appreciated by the skilled artisan (e.g., medical or familial history). Dose amount, frequency or duration may be increased or reduced, as indicated by the clinical outcome desired, status of the symptom(s) or pathology, and any adverse side effects of the treatment or therapy. The skilled artisan will appreciate the factors that may influence the dosage, frequency, and timing required to provide an amount sufficient or effective for providing a prophylactic or therapeutic effect or benefit. The exact dosage will be determined by the practitioner, in light of factors related to the subject that requires treatment. Dosage and administration are adjusted to provide sufficient levels of the active agent(s) or to maintain the desired effect.

The dosage may range broadly, depending upon the desired effects and the therapeutic indication. Alternatively, dosages may be based and calculated upon the per unit weight of the patient, as understood by those of skill in the art. In instances where human dosages for compounds have been established for at least some condition, those same dosages may be used, or dosages that are between about 0.1% and 500%, more preferably between about 25% and 250% of the established human dosage. Where no human dosage is established, as will be the case for newly discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

Dosage amount and interval may be adjusted individually to provide plasma levels of the active protein, which are sufficient to maintain the modulating effects, or minimal effective concentration (MEC) thereof. For example, therapeutic dosages of follistatin may result in plasma levels of 0.05 mg/mL, 0.1 mg/mL, 0.5 mg/mL, 1 mg/mL, 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, a range bounded by any two of the aforementioned numbers, or about any of the aforementioned numbers and ranges. As another example, therapeutic dosages of telomerase may result in plasma levels of 100 mg/mL, 150 mg/mL, 200 mg/mL, 250 mg/mL, 300 mg/mL, 350 mg/mL, 400 mg/mL, 450 mg/mL, 500 mg/mL, 550 mg/mL, 600 mg/mL, a range bounded by any two of the aforementioned numbers, or about any of the aforementioned numbers and ranges. In some embodiments, the therapeutic dose is sufficient to establish plasma levels in the range of about 250 mg/mL to about 400 mg/mL. The MEC may vary for each compound but can be estimated from in vitro or ex vivo data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration. It should be appreciated that the desired serum concentration of target protein may be adjusted or dosed according to the total number of viral genomes per kg of patient body weight required to reach the desired serum concentration.

As described herein, the methods of the embodiments also include the use of a compound or compounds as described herein together with one or more additional therapeutic agents for the treatment of aging-related disorders or conditions. Thus, for example, the disclosed vectors may be: (1) co-formulated and administered or delivered simultaneously in a combined formulation; (2) delivered by alternation or in parallel as separate formulations; or (3) by any other combination therapy regimen known in the art. When delivered in alternation therapy, the methods described herein may comprise administering or delivering the active ingredients sequentially (e.g., in separate solution, emulsion, suspension, tablets, pills or capsules) or by different injections in separate syringes. In general, during alternation therapy, an effective dosage of each active ingredient is administered sequentially (e.g., serially), whereas in simultaneous therapy, effective dosages of two or more active ingredients are administered together. Various sequences of intermittent combination therapy may also be used.

As used herein, all numerical values or numerical ranges include integers within such ranges and fractions of the values or the integers within ranges unless the context clearly indicates otherwise. Thus, to illustrate, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

As used herein, “co-administration” means concurrently or administering one substance followed by beginning the administration of a second substance within 2 hours, 20 hours, 16 hours, 12 hours, 8 hours, 4 hours, 1 hour, 30 minutes, 15 minutes, 5 minutes, 1 minute, a range bounded by any two of the aforementioned numbers, and/or about any of the aforementioned numbers. In some embodiments, co-administration is concurrent.

Some embodiments are directed to the use of companion diagnostics to identify an appropriate treatment for the patient. A companion diagnostic is an in vitro diagnostic test or device that provides information that is highly beneficial, or in some instances essential, for the safe and effective use of a corresponding therapeutic composition. Such tests or devices can identify patients likely to be at risk for adverse reactions as a result of treatment with a particular therapeutic composition. Such tests or devices can also monitor responsiveness to treatment (or estimate responsiveness to possible treatments). Such monitoring may include schedule, dose, discontinuation, or combinations of therapeutic compositions. In some embodiments, the therapeutic gene is selected by measuring a biomarker in the patient. The term biomarker includes, but is not limited to, genetic elements (e.g., expression level of a genetic element) or proteins (e.g., increase/decrease in expression level of a protein or concentration within a specific tissue or organ).

### EXAMPLES

### Example 1: Cells, Media, and Viruses

An MCMV bacterial artificial chromosome (MCMV-BAC) Smith strain was used. Mouse fibroblast 3T3 cells were used for the MCMV culture and growth assays and were cultured in minimal essential media (MEM) with 10% fetal bovine serum (FBS), 1% penicillin and streptomycin (P/S) at 37° C., 5% CO₂. Approved Institutional Biosafety Committee (IBC) and IACUC protocol were followed.

### Example 2: Construction and Characterization of Recombinant MCMV_{Luc}-_{TERT} and MCMV_{Luc}-_{FS344}

Recently, the CMV vector has emerged as a potential delivery vector for expressing therapeutic molecules, including proteins. CMV can infect different cell types in the body and is thus able to deliver the target proteins to numerous cell types. More specifically, CMV can infect fibroblast, hepatocytes, endothelial cells, macrophages, epithelial cells, lymphocytes, retinal pigment epithelial cells, and cells of the gastrointestinal tract. Therefore, CMV can infect and deliver its target antigens to different cell types in various organs of the body. Moreover, CMV can overcome the pre-existing immunity to efficiently express the protein of interest. Furthermore, CMV has a large genome size owing to its tremendous capacity to incorporate multiple genes and express them simultaneously.

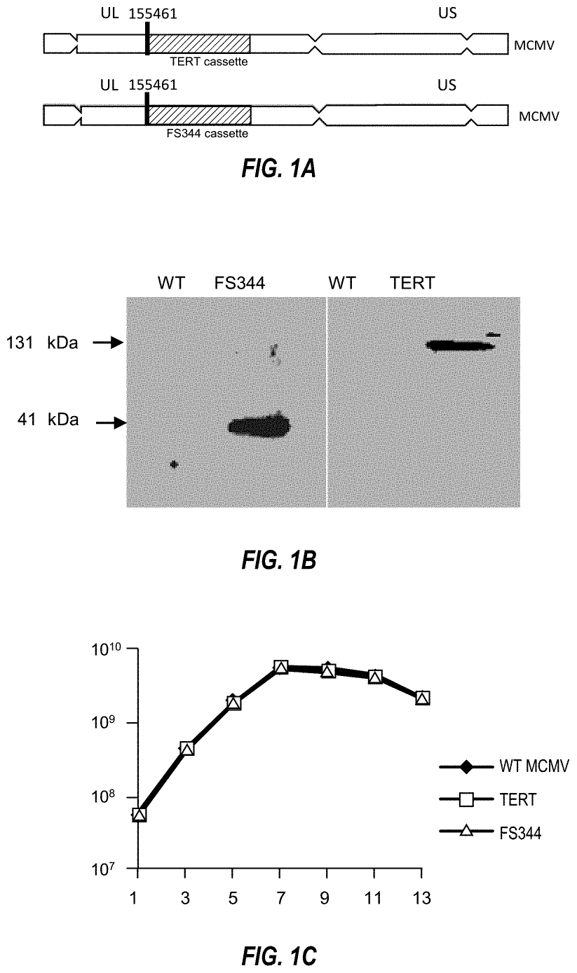

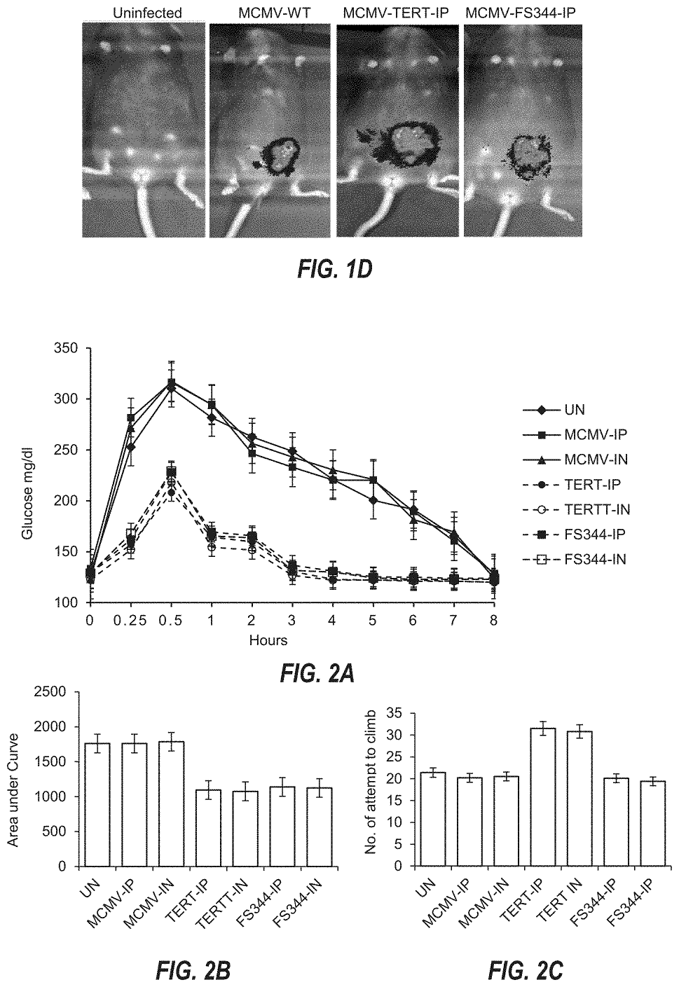

A BAC engineering method was used to generate a recombinant MCMV containing mouse telomerase reverse transcriptase (mTERT) gene as described previously (see Qiyi Tang, B. S., Hua Zhu. Protocol of a Seamless Recombination with Specific Selection Cassette in PCR-Based Site-Directed Mutagenesis. Applied Biological Engineering—Principles and Practice, 461-478 (2012). Similarly, a recombinant MCMV containing the mouse follistatin (mFS344) gene was generated (FIG. 1A). The plasmids containing the mouse TERT gene (MR226892) and mouse follistatin gene (MR225488) were obtained from Origene. The mTERT and mFS344 were fused with a FLAG-tag at the C-terminus. The strategy to generate the recombinant MCMV is shown in FIG. 1A. A CMV promoter was used to express the mTERT and mFS344 genes due to its strong transcriptional activity. The mTERT and mFS344 expression cassette were inserted at M107 locus of the MCMV_{Luc} genome without any deletion using a single-step integration event. The integration of mTERT and mFS344 into the MCMV genome was confirmed by PCR, Western blot, and immunofluorescence assay. The recombinant MCMV_{Luc}-_{TERT} and MCMV_{Luc}-_{FS344} were characterized by Western blot using FLAG-tag monoclonal antibody to check the expression of mTERT and mFS344 in the infected cells.

The recombinant MCMV_{Luc} vector was characterized by growth curve, and Western blot. A growth curve was performed in mouse fibroblast 3T3 cells. The cells in triplicate in a six-well plate were infected with 0.1 MOI of MCMV_{Luc}-_{TERT} and MCMV_{Luc}-_{FS344}. The growth curves were determined by measuring the relative luciferase unit (RLU) every alternate day using luciferin substrate by In vivo Imaging System (IVIS™ 50, Xenogen). Briefly, 3T3 cells were infected with the MCMV_{Luc}-_{TERT} and MCMV_{Luc}-_{FS344} cells at an MOI of 0.1. Two days later, the infected cells were harvested for Western blot analysis. Western blot results confirmed the expression of mTERT and mFS344 in the infected cells (FIG. 1B).

For Western blot analysis, 3T3 cells were infected with the MCMV_{Luc}-_{TERT} and MCMV_{Luc}-_{FS344} cells at an MOI of 0.2. Two days later, the infected cells were harvested and cell lysates were separated on 10% sodium dodecyl sulfate-polyacrylamide gel electrophoresis (SDS-PAGE) and proteins were transferred onto a PVDF membrane (Bio-Rad). The membrane was blocked with 3% BSA in 1× PBS for 2 hours at room temperature (RT) followed by 1-hour incubation with a mouse FLAG-tag antibody (1:2000 dilution in 3% BSA in 1× PBS). The membrane was washed three times with the 0.1% Tween-20 for 10 minutes each. Thereafter, the membrane was incubated with a secondary anti-mouse HRP conjugated antibody at a dilution of 1:10,000 in 1× BSA for 1 hour at RT. The blot was developed by chemiluminescence substrate (Bio-Rad).

Growth of MCMV_{Luc}-_{TERT} and MCMV_{Luc}-_{FS344} were determined using a luciferase assay. The growth curve analysis confirmed that MCMV_{Luc}-_{TERT} and MCMV_{Luc}-_{FS344} are similar to MCMV-WT and do not show any growth defect (FIG. 1C). The in vivo replication of recombinant viruses in 19-month old C57BL/6J mice also confirms that these viruses replicate as well as WT virus (FIG. 1D).

### Example 3: In Vivo Efficacy of MCMV_{Luc}-_{TERT} and MCMV_{Luc}-_{FS344} Recombinant Viruses in Old Mice

The therapeutic efficacy and safety of the disclosed and tested recombinant viruses was determined using 18-month old C57BL/6J mice. Two routes of viral administration were chosen in mice, namely intranasal (IN) and intraperitoneal (IP). The mice were separated into the following 7 groups with 9, 18-month old C57BL/6J mice per group: (1) MCMV_{Luc}-_{WT-IN}, (2) MCMV_{Luc}-_{WT-IP}, (3) Uninfected, (4) MCMV_{Luc}-_{TERT-IP}, (5) MCMV_{Luc}-_{TERT-IN}, (6) MCMV_{Luc-FS344-IP}, and (7) MCMV_{Luc}-_{FS344-IN}. The mTERT and mFS344 expressing recombinant viruses were injected once a month, at a dose of 1×10⁵ PFU/mouse.

### Example 4: Glucose Intolerance Test

Three mice from each group were selected at 22 months of age: (1) MCMV_{Luc}-_{WT-IN}, (2) MCMV_{Luc}-_{WT-IP}, (3) Uninfected, (4) MCMV_{Luc}-_{TERT-IP}, (5) MCMV_{Luc}-_{TERT-IN}, (6) MCMV_{Luc-FS344-IP}, and (7) MCMV_{Luc}-_{FS344-IN}. The mice were starved for 15 hours, followed by intraperitoneal injection with a 50 mg glucose solution. Blood samples were collected at 0 min, 15 min, 30 min, 60 min, 120 min, 180 min, 240 min, 300 min, 360 min, 420 min, and 480 min via a small incision in the tail vein of the mice. The blood glucose level was immediately determined using OneTouch Ultra glucose strips.

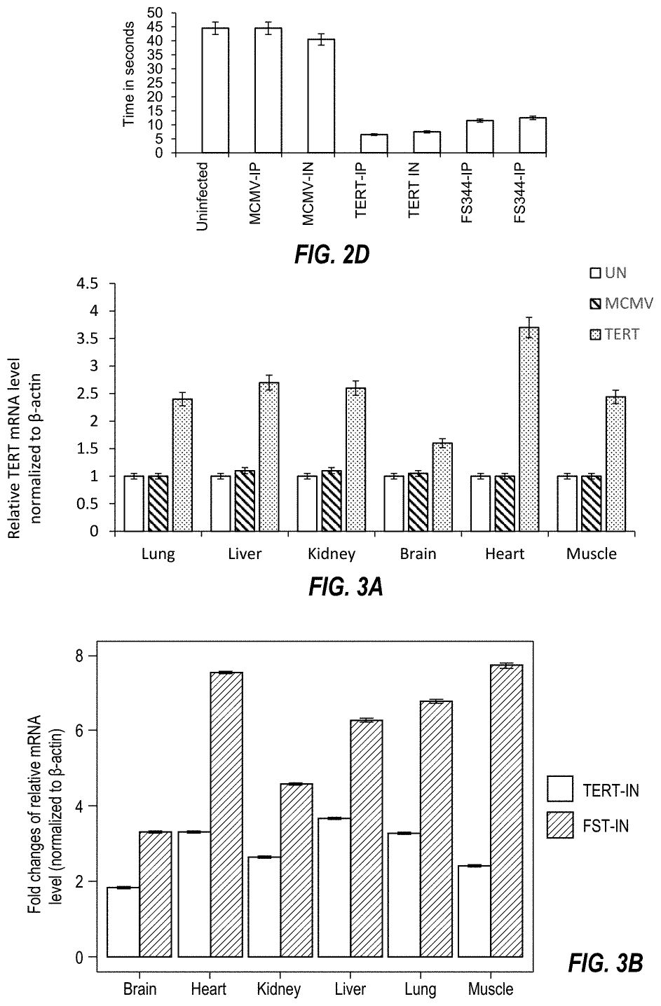

A peak of glucose level was observed at 30 minutes of glucose administration (FIG. 2A). After 30 minutes, the uninfected or WT treated mice were showing high blood glucose with an average value of 310 mg/dl and 316 mg/dl respectively, whereas the mTERT and mFS344 treated mice were showing 15 mg/dl and 165 mg/dl glucose. Moreover, blood glucose level came back to the normal basal level in 180 minutes in the mFS344 and mTERT treated mice. The WT or uninfected mice took about 480 minutes to bring blood glucose levels to the normal basal level (FIG. 2A). The area under curve (AUC) of the glucose curve is shown in FIG. 2B and confirms glucose intolerance of mice treated with mTERT and mFS34 recombinant viruses.

### Example 5: MCMV_{Luc}-TERT and MCMV_{Luc}-FS344 Infected Old Mice have Less Glycosylated Hemoglobin

The serum level of glycosylated hemoglobin is a direct measure of defective metabolism and indicated diabetic mice. Serum from mice in each group was analyzed by the ELISA kit (M0476F041, Mybiosource) per the manufacturer's method to calculate the level of glycosylated hemoglobin.

The level of glycosylated hemoglobin is a direct measure of blood glucose level and its increase is indicative of diabetes. Here, we measured the level of glycosylated hemoglobin in 23 month old mice after 5 consecutive administrations of respective therapy treatments. Mice infected with recombinant mTERT and mFS344 virus were found to contain less glycosylated hemoglobin in their blood as compared to the control or untreated mice. A high level of glycosylated hemoglobin is associated with diabetic conditions. This finding correlates well with the glucose tolerance test results, further supporting why MCMV_{Luc}-_{TERT} and MCMV_{Luc}-_{FS344} reduce the blood glucose over 3 times faster than the control groups.

### Example 6: MCMV_{Luc}-_{TERT} and MCMV_{Luc}-_{FS344} Treated Old Mice have Increased Activity and Coordination

The bodyweight of all mice in each group was measured and recorded twice a month. An increase in body mass was considered to be a measure of accumulation of more myocytes in the body. The mice in each set were analyzed by postmortem for the muscle gain.

Body activity and coordination are decreased progressively with increasing age. A measure of activity was considered an indication of the overall health of a patient. Therefore, a beaker test was performed to determine the activity of the treated elderly mice in each group. This test was performed when mice were 23-months old. Three mice from each group were put in a one liter glass beaker and general activity and attempts to climb out of the beaker were measured. The protocols were properly followed as described previously (see, for example, Magno, et al. Optogenetic Stimulation of the M2 Cortex Reverts Motor Dysfunction in a Mouse Model of Parkinson's Disease. J Neurosci 39, 3234-3248, doi:10.1523/Jneurosci.2277-18.2019 (2019) and Magno, et al. Cylinder Test to Assess Sensory-motor Function in a Mouse Model of Parkinson's Disease. Bio-Protocol 9, doi:ARTN e3337 10.21769/BioProtoc.3337 (2019)).

MCMV_{Luc}-_{TERT} treated animals were more active than uninfected or MCMV_{Luc}-_{WT} treated mice (FIG. 2C). The MCMV_{Luc}-_{FS344} treated mice were bulkier and were not able to climb but showed increased activity by walking faster on the floor of the beaker than the controls. Surprisingly, mFS344 treated mice were more active in penetrating the bedding of cages than other groups, suggesting more power and energy.

Aging mice have reduced coordination and motor skills. The coordination of treated and untreated mice was analyzed using a beam walking assay as performed previously (see Luong, et al. Assessment of Motor Balance and Coordination in Mice using the Balance Beam. Jove-J Vis Exp, doi: 10.3791/2376 (2011)). In short, three, 23-month old mice from each group were trained to traverse a 4 ft long, 10 mm wide beam for two consecutive days, then tested on the third day. Results were obtained as the amount of time taken by each mouse to cross the beam. The mice treated with MCMV_{Luc}-_{TERT} and MCMV_{Luc}-_{FS344} have better coordination as compared to the uninfected or MCMV_{Luc}-_{WT} treated mice (FIG. 2D). These results confirmed that MCMV_{Luc}-_{TERT} and MCMV_{Luc}-_{FS344} treated mice have improved cognitive ability paired with enhanced activity and coordination, suggesting improved health in aging mice.

### Example 7: Tissues RNA Isolation, cDNA Preparation and Real-Time PCR

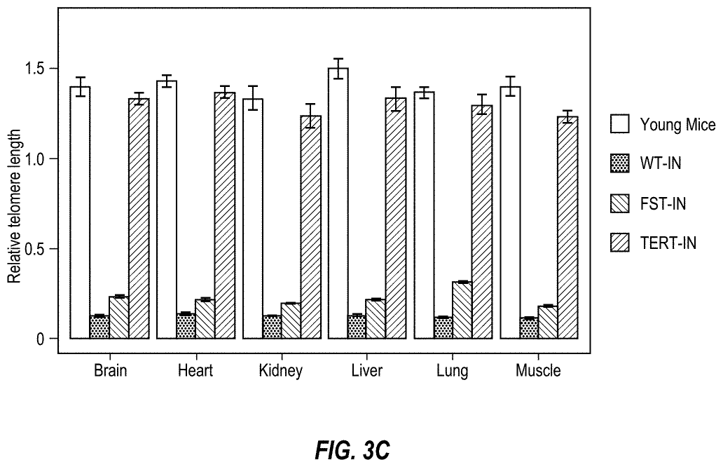

To determine the level of mTERT and mFS344 in various tissues of mice infected with MCMV_{Luc}-_{TERT}, real-time PCR was performed on cDNA prepared from RNA of infected tissues. Eleven-month old mice (3 in each group) were infected with wild type MCMV, MCMV_{Luc}-_{TERT}, and MCMV_{Luc}-_{FS344}. The heart, brain, lung, liver, and kidney were isolated 6-days post infection. Uninfected mice were used as a control. The tissues were homogenized, and RNA was isolated using RNeasy® Mini Kit (Qiagen). Purified RNA concentration was determined by measuring optical density. Approximately, 1.0 μg of RNA was used to prepare cDNA using a Titanium RT-PCR kit (TaKaRa). The real-time PCR was performed on the cDNA using mTERT, and mFS344 primers as described previously. β-actin was used as an internal control for normalization. The expression of mTERT and mFS344 was determined in respective tissues after normalization with β-actin. Results for TERT mRNA levels are shown in FIG. 3A. The fold increase in mRNA levels for TERT and FST are shown in FIG. 3B. As shown, TERT mRNA was increased by ˜1.9, 3.3, 2.7, 3.7, 3.3, and 2. folds in brain, heart, kidney, liver, lung, and muscle respectively as compared to the control, while FST mRNA was increase by ˜3.3, 7.6, 4.6, 6.3, 6.8, and 7.8 folds in brain, heart, kidney, liver, lung, and muscle respectively as compared to the control (FIG. 3B).

### Example 8: Determination of Relative Telomere Length in Organs of Treated and Untreated Mice

FIG. 3C illustrates the determination of relative telomere length in different organs (e.g., heart, brain, liver, kidney, lung and muscle) of treated and untreated groups at 24-months-old mice. An 8-months-old mouse was also measured. The tissues were homogenized and genomic DNA was isolated using Sigma genomic DNA isolation kit (GeneElute genomic DNA isolation kit). The qPCR was performed on the genomic DNA using specific telomeric primers. A pair of 36B4 gene primers was used as a single copy gene for normalization. The relative telomere length was calculated by a Δ CT value as described previously. (M. V. Joglekar et al., An Optimised Step-by-Step Protocol for Measuring Relative Telomere Length. Methods Protoc 3 (2020)). Telomere length was determined in kidney and muscle using Cy3 PNA probes and Q-FISH. Briefly, kidney and muscles were harvested from 24-months-old mouse sacrificed in each group and processed for histology sectioning as described previously. (M. M. Tong et al., Mitophagy Is Essential for Maintaining Cardiac Function during High Fat Diet-Induced Diabetic Cardiomyopathy. Circ Res 124, 1360-1371 (2019)). The Cy3 labeled peptide nucleic acid (PNA) probe for fluorescence in situ hybridization (FISH) was acquired from PAN BIO (catalogue #F1002). The telomere staining was performed as per the manufacturer instruction (PNA BIO, F1002). Z-stacks images were acquired using Axiovert 200M microscope. The relative telomere length in each tissue was determined for each sample as described previously. (A. Suram et al., Oncogene-induced Telomere Dysfunction Enforces Cellular Senescence in Human Cancer Precursor Lesions. Embo J 31, 2839-2851 (2012); N. Razdan, T. Vasilopoulos, U. Herbig, Telomere Dysfunction Promotes Transdifferentiation of Human Fibroblasts into Myofibrobalsts. Aging Cell 17 (2018)).

As shown, the follistatin treated mice showed increased telomere length relative to untreated mice. The TERT treated mice showed even greater increases in telomere length relative to untreated mice. Surprisingly, the TERT treated mice showed telomere lengths approaching (or in some cases not statistically different from) the telomere lengths of the young (8-months-old) mice.

### Example 9: Serum Level of mTERT and mFS344 Protein in Old and Young Mice Infected with MCMV_{Luc}-_{TERT} and MCMV_{Luc}-_{FS344}

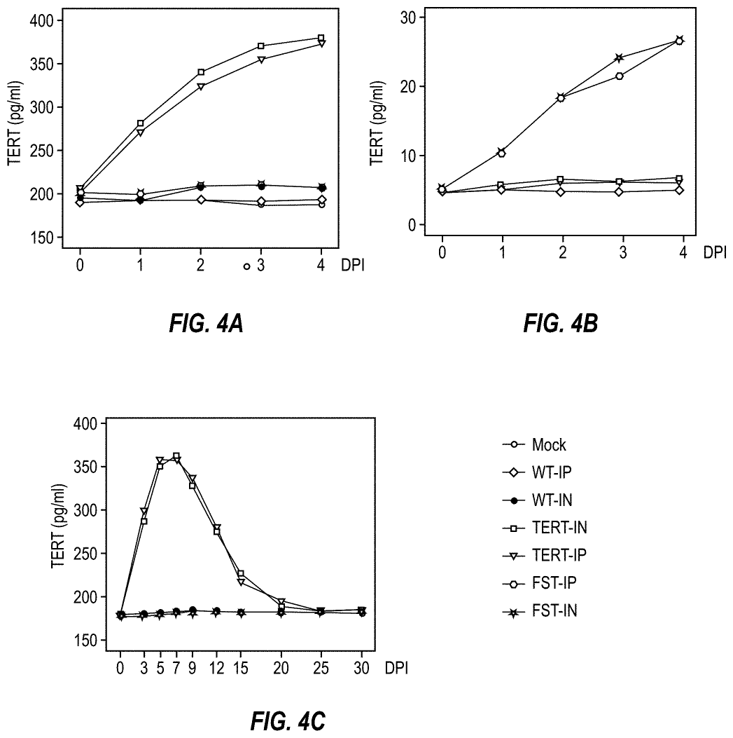

After 6 consecutive months of administration of recombinant MCMV_{TERT} and MCMV_{FS344} virus in 18-month old mice, blood was analyzed for 3-4 days to determine the serum level of mTERT and mFS344. The collected blood was centrifuged and serum was separated. A mouse telomerase reverse transcriptase ELISA kit (MBS1601022, Mybiosource) and a mouse follistatin ELISA kit (MBS1996306, MyBioSource) were used to measure the serum level of mTERT and mFS344, respectively. The protocol and instructions were followed as per the manufacturer's method. ELISA results demonstrate that there was an increase in the level of mTERT (380 pg/ml) and follistatin (26.6 ng/ml) in the serum after the administration of recombinant viruses compared to the control or MCMV infected (“Mock” treated) mice (FIGS. 4A & 4B). It confirms that MCMV_{Luc}-_{TERT} and MCMV_{Luc}-_{FS344} infect various cells types and express the target proteins.

The experiment was repeated with 8-month old C57BL/6J mice to determine the kinetics of mTERT and mFS344 expression over one month (FIG. 4C). Three mice in each group were infected with MCMV_{Luc}-_{TERT} and blood samples were collected at indicated time points. The serum mTERT level was highest (˜400 ng/mL) at 5-7 days post-infection. After 7 days of infection, the expression of mTERT decreases gradually, reaching basal level by day 25 (FIG. 4C).

### Example 10: MCMV_{Luc}-_{TERT} and MCMV_{Luc}-_{FS344} Infected Old Mice Look Younger with Less Hair Loss

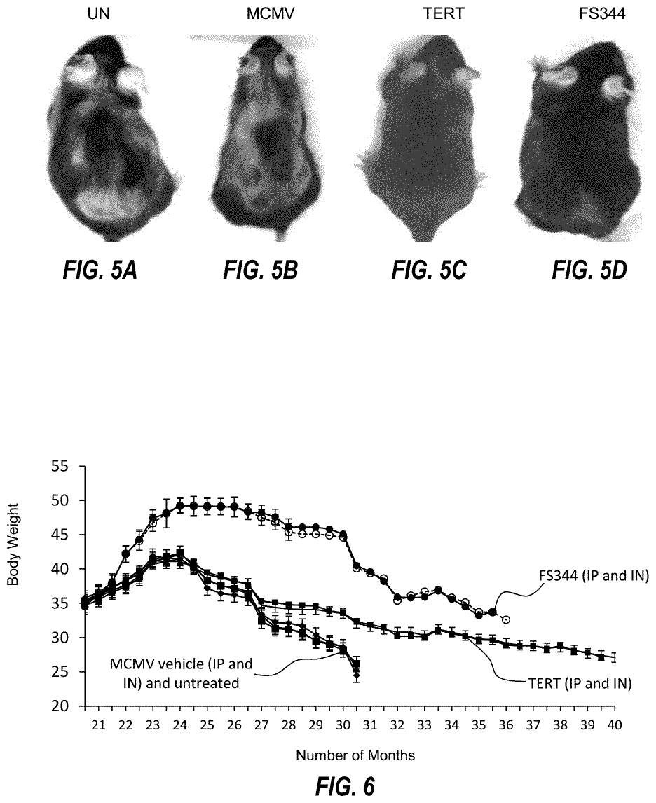

Hair loss was observed in each group of mice. MCMV_{Luc}-_{TERT} and MCMV_{Luc}-_{FS344} treated mice were observed for body hair loss over time. The mice treated with a wild-type MCMV vector or left untreated were used as controls. High-resolution photographs were captured and analyzed in Photoshop to determine the degree of hair loss in each mouse. The pictures in FIGS. 5A-5D are of 24-month old mice that are a representative specimen of their respective groups.

MCMV_{Luc}-_{TERT} treated animals were resistant to hair loss and retained 99 percent of their body hair after 26 months. At this time point, the mice in each group were administered with respective virus 8 times for 8 months of anti-aging therapy. The untreated and MCMV_{Luc}-_{WT} treated mice were losing their hair rapidly and retained only 40 percent of the hair (FIG. 5B). MCMV_{Luc-TERT} (FIG. 5C) and MCMV_{Luc}-_{FS344} (FIG. 5D) treated animals were looking younger as compared to the uninfected (FIG. 5A) and MCMV_{Luc}-_{WT} (FIG. 5B) treated animals and did not lose their hair after a period of 26 months. These results strongly suggest that mice infected with MCMV_{Luc}-_{TERT} and MCMV_{Luc}-_{FS344} were more resistant to hair loss and look younger, while the mice in untreated or MCMV_{Luc-WT} treated cages were looking old and were losing their hair continuously with age. The loss in hair may be correlated with the TERT expression in the skin cells, as some studies have hinted at an increase in TERT expression within skin as potentially facilitating hair growth by enhancing the follicle stem cells proliferation. Unexpectedly, however, the mice infected with recombinant MCMV_{Luc}-_{FS344} virus also demonstrated a similar retention of hair as the MCMV_{Luc}-_{TERT} infected mice.

### Example 11: MCMV_{Luc}-_{TERT} and MCMV_{Luc}-_{FS344} Infected Old Mice have Increased Bodyweight

Recombinant viruses were injected once a month into each group of 18-month old C57BL/6J mice to determine therapeutic efficacy. The bodyweight of all mice in each group was measured twice a month to see any effect of recombinant viruses on normal health. An increase in body mass was interpreted as accumulation of myocytes and increase in musculature. MCMV_{Luc}-_{FS344} treated mice weighed ˜40% more than MCMV_{Luc}-_{WT} treated mice. The average body weight of MCMV_{Luc}-_{FS344} treated mice was ˜49 gm after 150 days of treatment (FIG. 6). The uninfected and MCMV_{Luc}-_{WT} treated mice have an average weight of ˜35 gm and ˜37 gm after 150 days of treatment. The MCMV_{Luc-TERT} infected mice had an average body weight of ˜39 gm and showed an overall resistance in body weight loss (FIG. 6). Bodyweight analysis suggested an increase in body weight for MCMV_{Luc}-_{FS344} treated mice as compared to MCMV_{Luc}-_{WT} treated or uninfected groups. The MCMV_{Luc-TERT} infected animals have substantially gained bodyweight and showed resistance in losing body weight with age.

Administration of MCMV_{Luc-TERT} and MCMV_{Luc}-_{FS344} was stopped after mice reached 29 months of age. Surprisingly, mice started losing weight (FIG. 6). This could be due to waning expression of mTERT and mFS344. After 32 months, therapy was reinitiated and an increase in body weight was measured, likely due to the expression of mFS344gene (FIG. 6). These results suggest a dose dependent increase in bodyweight of mice infected with MCMV_{Luc}-_{FS344}

### Example 12: MCMV_{Luc}-_{TERT} and MCMV_{Luc}-_{FS344} Treated Old Mice have Increased Life Span

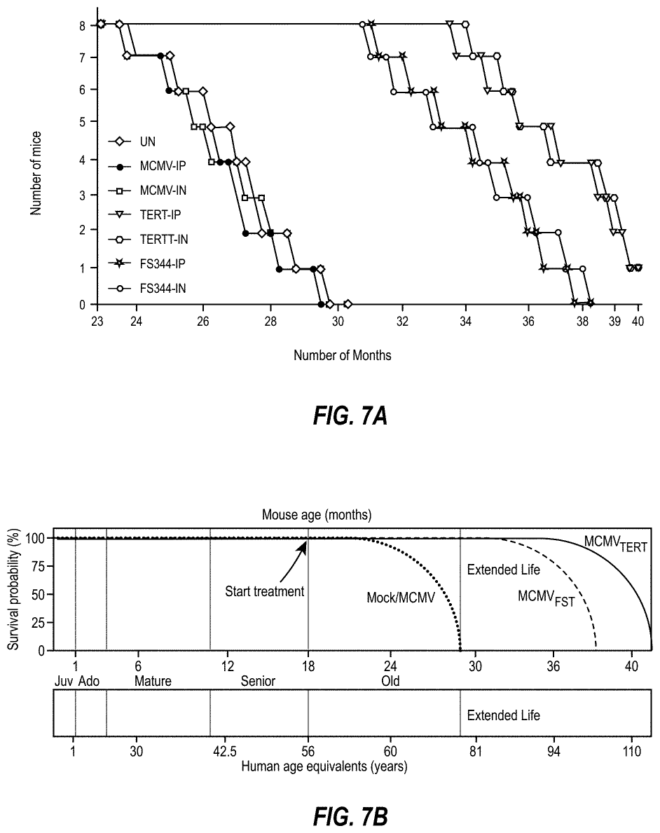

A Kaplan-Meier test was used to determine the living and dead MCMV_{Luc-TERT} and MCMV_{Luc}-_{FS344} treatment subjects at a given interval of time. The control animals died earlier than MCMV_{Luc}-_{FS344} or MCMV_{Luc-TERT} animals. A significant life extension in MCMV_{Luc}-_{TERT} and MCMV_{Luc}-_{FS344} treated mice was observed as compared to the control. The average age of mice in MCMV_{Luc-WT-IP}, MCMV_{Luc-WT-IN}, and the uninfected group were 26.4, 26.6, and 26.6 months, respectively (FIGS. 7A and 7B). The average age of mice in MCMV_{Luc}-_{FS344-IN} and MCMV_{Luc}-_{FS344-IP} was 34.3 and 34.6 months, respectively (FIGS. 7A and 7B). MCMV_{Luc}-_{TERT-IN} and MCMV_{Luc}-_{TERT-IP} treated mice have an average age of 37.1 and 37.3 months, respectively (FIGS. 7A and 7B). A percentage increase in the longevity of mice in MCMV_{Luc}-_{TERT} (41.1%) and MCMV_{Luc}-_{FS344} (30.3%) infected groups was determined compared with the MCMV_{Luc-WT} infected or untreated mice. A significant increase in the median age of the mice in MCMV_{Luc}-_{TERT} and MCMV_{Luc}-_{FS344} treated mice was also observed as compared to the control groups (FIGS. 7A and 7B).

These results demonstrate that MCMV_{Luc}-_{FS344} treated mice have a 30.3 percent increase in age longevity as compared to the control or untreated mice (FIGS. 7A and 7B). The mice infected with MCMV_{Luc}-_{TERT} has a 40.1 percent increase in the age as compared to MCMV_{Luc-WT} infected or untreated groups (FIGS. 7A and 7B). These results strongly indicate overall longevity for MCMV_{Luc}-_{TERT} and MCMV_{Luc}-_{FS344} treated mice as compared to the uninfected or MCMV_{Luc}-_{WT} treated mice. The significant increase in longevity with increased activity and coordination indicated improved health in old mice. The CMV vector is able to infect a wide variety of cells in the body and thus is able to deliver the mTERT and mFS344 genes that lead to the significant increase in life span with increased coordination and body activity.

### Example 13: Old Mice Treated with MCMV_{Luc}-_{TERT} and MCMV_{Luc}-_{FS344} have Higher Mitochondria Number with Increased Cristae Surface Area in Heart and Skeletal Muscle Tissue

It has been demonstrated previously that accelerated aging produced by shortening of telomeres over time involves mitochondrial deterioration, suggesting a connection between telomerase and mitochondria in the aging process. Mitochondrial dysfunction is associated with aging, as cellular metabolism is impaired.

To observe changes occurring at the cellular level, electron microscopy was performed of tissue samples collected from animals in each set. One mouse from each group was sacrificed after 6 consecutive months of recombinant virus administration, and the heart and skeletal muscles were isolated. The tissue samples were fixed in EM buffer and kept at 4° C. until processed. Conventional EM was performed as known in the art.

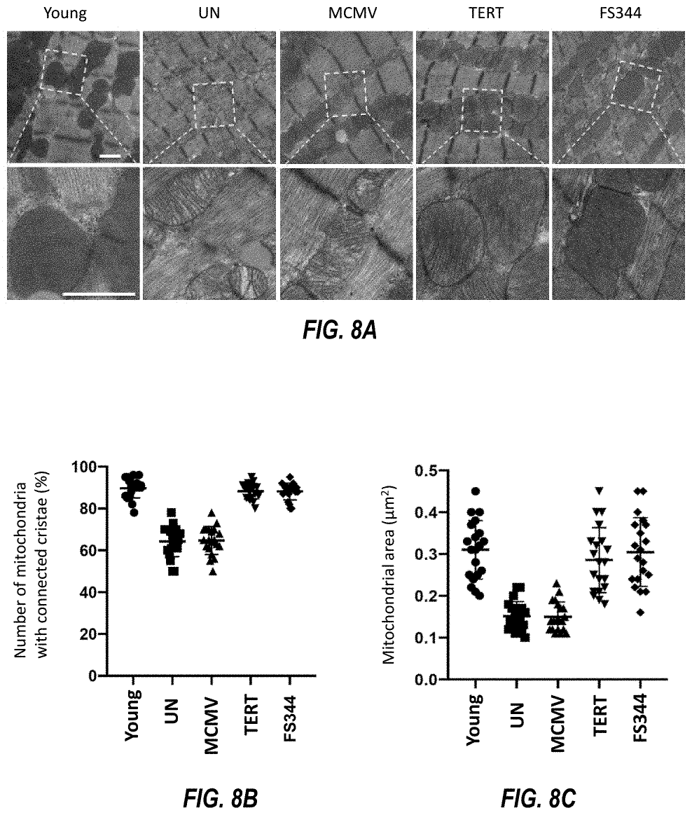

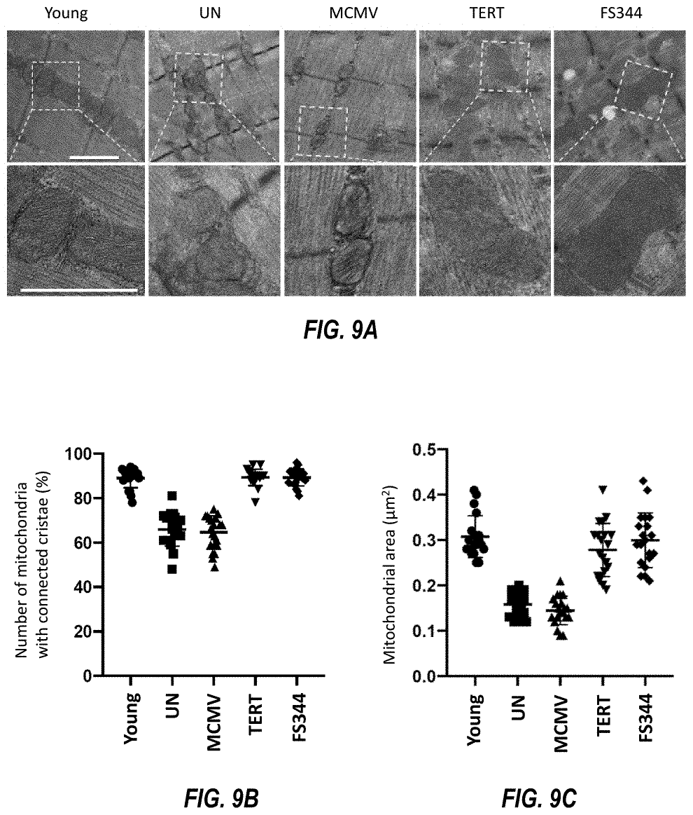

The electron microscopy analysis of heart and skeletal muscle tissues from MCMV_{Luc-TERT} and MCMV_{Luc-mFS344} treated mice have an increased number of mitochondria with connected cristae in heart and skeletal muscle tissue, comparable to 6 months old controls (FIGS. 8A, 8B, 9A, and 9B). The mice in the control group (MCMV and UN) have fewer mitochondria overall (FIGS. 8A, 8B, 9A, and 9B). The calculated surface area of heart tissue mitochondria in mice infected with MCMV_{Luc}-_{TERT} and MCMV_{Luc}-_{FS344} increased and was comparable to the young mice (FIGS. 8C and 9C).

### Example 14: Autophagy and Antiaging Markers

To address the mechanism behind the observed increased number mitochondria and their surface area, the level of PGC1α, a transcription coactivator required for the proper mitochondrial function and regulation of cellular signaling pathways controlling cell aging, was measured. Mitochondria and autophagy play a critical role in the biological aging process. The accumulation of dead or aging cells leads to cellular and systemic dysfunction, and thus causes aging. To determine the exact changes in the body of treated old mice, the protein level of various integral markers of autophagy and aging were measured. Western blot was used to determine the level of PGC1α (Novus Bio, NBP1-04676), Complex I, II, III, and V (Thermofisher, 45-8099), TFAM (Abcam, ab131607), LC3 (BML,M183-3), and p62 (Abcam, ab91526) markers in the tissue homogenates of treated and control mice. The specific monoclonal antibodies were used for specific markers.

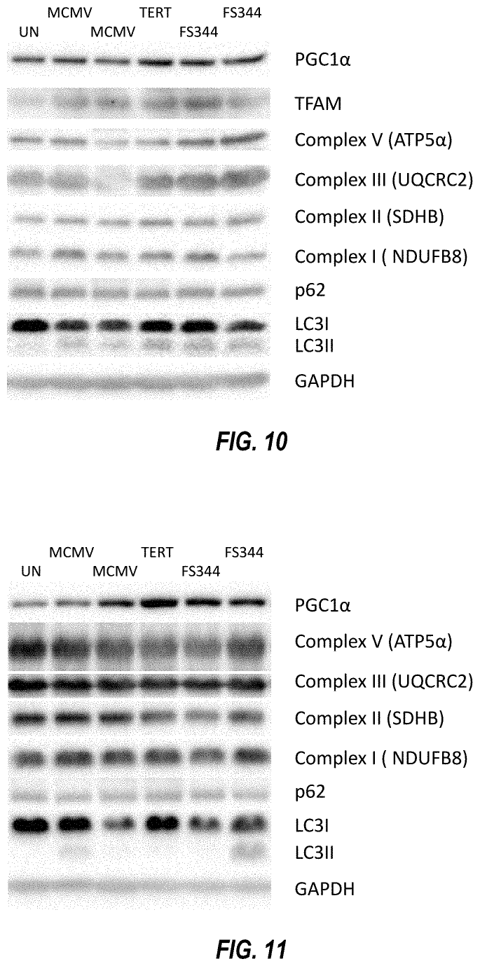

The MCMV_{Luc-TERT} and MCMV_{Luc-FS344} treated mice show increased autophagy markers by Western blot, suggesting the active removal of aging cells at a faster rate than the control group. The protein level of Complex I, II, III and V, PGC1α, TFAM, LC3I, LC3II, and p62 markers were specifically determined in the skeletal muscle tissue homogenates of treated and control mice (FIG. 10). The level of coactivator transcription factor PGC1α and TFAM (mitochondria transcription factor A) was found increased in the skeletal muscles as compared to the control and untreated mice, suggesting increased mitochondrial biogenesis and other cellular pathways associated with muscle fiber development (FIGS. 10 and 11). An increased level of complex III (UQCRC2) in MCMV_{Luc-TERT} and MCMV_{Luc-FS344} treated mice was observed as compared to the control or untreated groups (FIGS. 10 and 11). Complex III is an essential component of mitochondria and is involved in ATP synthesis as well as oxygen homeostasis.

### Example 15: MCMV_{Luc}-_{TERT} and MCMV_{Luc}-_{FS344} Treated Old Mice did Not Show Incidence of Cancer

Because TERT activity increases in cancer cells, there is a concern that induced overexpression of mTERT may increase the risk of tumor or cancer development. However, no malignancies were detected upon autopsy in either treated group. Additionally, ante mortem daily observations did not reveal any other defects such as paralysis, body dysfunctions, or blindness. Notably, re-expressing telomerase in a model of increased aging could not detect cancer. These results demonstrate that overexpression of mTERT in old mice in the context of a recombinant CMV vector does not cause any cancer phenotype or malignancies, nor any other obvious systemic dysfunction.

In conclusion, mice infected with the recombinant TERT or FS344 virus show an increase in body weight, high glucose tolerance, high serum level of TERT and FS344, increased activity, and other anti-aging properties. The treated mice have younger mitochondria in heart and muscle tissues and an increased level of PGC1α which controls the expression of genes that cause aging. Moreover, the treated mice show an increased level of complex III, V, TFAM, and 1C3II leading to the anti-aging effect on old mice. These results demonstrate that CMV is an excellent vector for delivering the therapeutic TERT and/or FS344 proteins in patients and has immense potential for gene therapy.

What is claimed is:

1. A recombinant CMV viral vector comprising one or more exogenous donor genes selected from a telomerase reverse transcriptase (TERT) gene, a follistatin-344 (FST) gene, a klotho (KL) gene, a damage suppressor (Dsup) gene, a peroxisome proliferator-activated receptor gamma coactivator 1-alpha (PGC-1-α) gene, an octamer-binding transcription factor 4 (Oct-4) gene, a sex determining region Y-box 2 (Sox2) gene, and a Krüppel-like factor 4 (KLF4) gene.

2. The recombinant CMV viral vector of claim 1, wherein the one or more exogenous donor genes are selected from TERT, FST, KL, Dsup, and PGC-1-α.

3. The recombinant CMV viral vector of claim 1, wherein the TERT gene is the human telomerase reverse transcriptase (h IERT) gene.

4. The recombinant CMV viral vector of claim 1, wherein the FST gene is the human follistatin-344 (hFST) gene.

5. The recombinant CMV viral vector of claim 1, wherein the one or more exogenous donor genes are fused to a native CMV gene selected from the IE1 gene, IE2 gene, pp65 gene, UL21.1 gene, UL21.5, gB gene, TRL4 gene, UL89 gene, US3 gene, R160461 gene, and R27080 gene.

6. The recombinant CMV viral vector of claim 5, wherein the one or more exogenous donor genes are fused to a native CMV gene selected from the IE1 gene, pp65 gene, and gB gene.

7. The recombinant CMV viral vector of claim 1, wherein the one or more exogenous donor genes are fused to a native CMV gene via a sequence coding for a 2A self-cleaving peptide.

8. The recombinant CMV viral vector of claim 7, wherein the 2A self-cleaving peptide is selected from T2A, P2A, E2A, and F2A.

9. The recombinant CMV viral vector of claim 8, wherein the 2A self-cleaving peptide is P2A.

10. The recombinant CMV viral vector of claim 1, wherein the viral vector comprises multiple exogenous donor genes.

11. The recombinant CMV viral vector of claim 10, wherein the viral vector comprises three exogenous donor genes.

12. The recombinant CMV viral vector of claim 1, wherein the viral vector comprises a bacterial artificial chromosome (BAC) in which the one or more exogenous donor genes are disposed.

13. The recombinant CMV viral vector of claim 1, wherein the CMV is a mouse CMV (MCMV), a primate CMV, or a human CMV (HCMV)

14. A method for treating, reversing, or preventing an age-related disorder or condition, comprising administering a therapeutically effective amount of the recombinant viral vector as in claim 1.

15. The method of claim 14, wherein the recombinant viral vector is administered via intranasal delivery or as an injectable therapeutic.

16. The method of claim 14, wherein administering the therapeutically effective amount of the recombinant viral vector prevents age-related hair loss.

17. The method of claim 14, wherein administering the therapeutically effective amount of the recombinant viral vector increases blood glucose tolerance.

18. The method of claim 14, wherein administering the therapeutically effective amount of the recombinant viral vector increases mitochondrial health, as measured by one or more of a percentage of mitochondria within skeletal or heart muscles having connected cristae or having an increased density of mitochondria compared to untreated age-matched averages.

19. A recombinant CMV viral vector comprising:
one or more exogenous donor genes selected from a human telomerase reverse transcriptase (hTERT) gene, a human follistatin-344 (hFST) gene, a klotho (KL) gene, a damage suppressor (Dsup) gene, a peroxisome proliferator-activated receptor gamma coactivator 1-alpha (PGC-1-α) gene, an octamer-binding transcription factor 4 (Oct-4) gene, a sex determining region Y-box 2 (Sox2) gene, and a Krüppel-like factor 4 (KLF4) gene,
wherein the one or more exogenous donor genes are fused to a native CMV gene selected from the IE1 gene, pp65 gene, and gB gene,
wherein the one or more exogenous donor genes are fused to a native CMV gene via a sequence coding for a 2A self-cleaving peptide, and
wherein the viral vector comprises a bacterial artificial chromosome (BAC) in which the one or more exogenous donor genes are disposed.

20. The recombinant CMV viral vector of claim 19, wherein the one or more exogenous donor genes are selected from TERT, FST, KL, Dsup, and PGC-1-α, and wherein the 2A self-cleaving peptide is P2A.
